(19) United States
(12) Patent Application Publication
(10) Pub. No.: US 2026/0248499 A1
(43) Pub. Date: Aug. 27, 2026

(54) HARDENING MATERIAL AND IMPLANT

(71) Applicant: Surgical Fusion Technologies GmbH, Schlieren (CH)

(72) Inventors: Jörg MAYER, Niederlenz (CH); Felix KRAEFT, Schlieren (CH); Rick SANER, Zürich (CH); Andrea MÜLLER, Winterthur (CH)

(21) Appl. No.: 19/551,328

(22) Filed: Feb. 26, 2026

Related U.S. Application Data
(60) Provisional application No. 63/763,652, filed on Feb. 26, 2025.

(51) Int. Cl. A61B 17/04; A61B 17/00; A61B 17/16; A61B 17/32; A61B 17/56; A61B 17/88

(52) U.S. Cl. CPC A61B 17/0401; A61B 17/1604; A61B 17/1655; A61B 17/32; A61B 17/8802; A61B 2017/00955; A61B 2017/0414; A61B 2017/0464; A61B 2017/564

(57) ABSTRACT

An implant for use together with a hardening material in the context of orthopedic applications and associated methods of implanting an implant in a hardening material.

### CROSS-REFERENCE TO RELATED APPLICATIONS

This application claims the benefit and priority to U.S. Provisional Application No. 63/763,652, filed Feb. 26, 2025, which is incorporated by reference herein in its entirety for all purposes.

### FIELD

The current invention relates to an implant for use together with a hardening material in the context of orthopedic applications.

### BACKGROUND

Hardening materials such as bone cements are widely used in orthopedic applications, for example to improve fixation of objects in or to bones, or to improve or restore bone structures. In the context of the present invention, the term “orthopedic” encompasses any type of treatment or surgery of any part of the musculoskeletal system, including any types of bones in humans or animals as well as osseous allografts and autografts or artificial grafts. A known type of hardening materials is based on acrylic polymers, such as methyl methacrylates, which provide a strong structure but are non-bioactive, non-resorbable and strongly exothermic (e.g. 50-90° C.) during polymerization, which can lead to damages on or around the bone, in particular to necrosis of the periosteum. Hence, the application of acrylic polymers is typically limited to large bones which can dissipate heat during the polymerization reaction. A further drawback of acrylic polymers is that they do not promote regeneration of damaged bones, are non-resorbable and do not convert to bone material. As an alternative to acrylic polymer-based cements, hardening materials such as (biodegradable or bioactive) ceramics, glasses and metals can be used in orthopedic applications. Known types of these materials include biodegradable (optionally hydraulic) cements, such as calcium- (e.g. calcium phosphate-) based cements which harden based on dissolution and precipitation reactions at moderate temperatures, e.g. around 37° C. Calcium-based cements, e.g. calcium phosphate-based cements (CPCs), are resorbable, bioactive and promote bone regeneration. However, a drawback of many hardening materials, and in particular of biodegradable cements, such as calcium-based cements including CPCs, is that they are brittle, porous and mechanically less stable, which limits, e.g., load-bearing applications.

### SUMMARY

It is a task of current invention to provide means and methods to address the drawbacks of hardening materials (e.g. biodegradable cements) in orthopedic applications, in particular the brittleness and mechanical stability of these hardening materials.

This task is solved by a kit of parts with the features of claim 1 and the methods of claims 10, 13 and 14. Further embodiments of the kit of parts and the methods are defined by the features of dependent claims.

In a first aspect, the present invention is directed to a kit of parts for implanting an implant into a hard tissue, comprising:
a hardening material, optionally a cement, optionally a hydraulic cement, in particular a calcium phosphate-based cement, for orthopedic use, optionally in powder or neat form, and
an implant having thermoplastic properties.

The definitions and explanations given for the terms and features of the present invention apply to all aspects and embodiments disclosed herein, unless specifically indicated otherwise.

The hardening material of use in the present invention can be any material that is suitable for orthopedic applications and that features load bearing and/or or load transferring properties when hardened. The hardening material can support or reinforce the bone material. Such hardening materials are known to the skilled person and include the materials disclosed in Liu et al. J. Funct. Biomater. 2023, 14, 134.

The hardening materials for use in the present invention are preferably inorganic materials, comprise inorganic materials or are based on inorganic materials. For example, the hardening materials can be non-polymeric materials (e.g. do not comprise polymeric materials). Preferably, the hardening materials for use in the present invention exclude materials that harden exothermically at temperatures higher than 50° C., but rather refer to materials that harden at temperatures of up to 45° C. or 50° C. The term “hardening” as used herein means that the material can harden at least to the extent that it exhibits load bearing and/or load transferring properties, and can include thixotropic materials, e.g. thixotropic gels. Exemplary thixotropic gels include gels comprising calcium-phosphate. For example, these gels can be used while ultrasound is applied to the gel, e.g. while introducing the gel into bone material, e.g. to support or reinforce trabecular structures.

Exemplary hardening materials include biodegradable cements, hydraulic or non-hydraulic cements, e.g. Ca-based cements, e.g. calcium phosphate-based cements, or CaCO₃ or CaSO₄-based cements, silicate-based ceramics, silicate, borate and phosphate glasses. In an example, the hardening material for use in the present invention does not comprise thermoplastic polymers. The hardening material, in particular hydraulic cements, may comprise additives which create pores in the material, such as water-soluble salts or hydrogels which swell and subsequently dissolve creating pores.

For example, hydraulic cements can be calcium phosphate-based cements (CPC). A CPC, as used herein, refers to any cement that comprises calcium phosphate and is suitable for orthopedic applications. Suitable CPCs are known in the art, and the skilled person can choose any CPC for the purposes of the present invention that is suitable for orthopedic use. For easier legibility, the word “cement” may be used herein which refers to a biodegradable, optionally hydraulic cement, in particular a CPC as mentioned above. Exemplary CPCs include calcium orthophosphate compounds such as, e.g. compounds including monocalcium phosphate monohydrate, dicalcium phosphate dihydrate, octocalcium phosphate, precipitated hydroxyapatite, calcium-deficient hydroxyapatite, amorphous calcium phosphate, and mixtures thereof. Exemplary CPCs are brushite-, monetite- or hydroxyapatite-based one, two or multicomponent cement systems; Calcium sulfate-based cements or organic-inorganic-composite cements containing a organic admixture (preferably not a thermoplast) that can be divided into natural polymers and synthetic polymers. Natural polymers mainly include gelatin, sodium alginate, silk fibroin and chitosan (see, e.g., P. G. Ginebra. Chapter 10 in Orthopedic bone cements, Sanjukta Deb (ed) Woodhead publishing, 2008 and Liu et al, J. Funct. Biomat. 2023, 14, 134)

As known to the skilled person, a hardening material and in particular a cement is typically stored in a dry form (e.g. water-free and/or solvent-free) such that it does harden, e.g. not form hardened hardening material, during storage. The kit of parts described herein preferably comprises the hardening material in a storable form, e.g. as a powder, neat, water-free and/or solvent-free, wherein optionally the hardening material is provided in a vacuumized container. However, the hardening material in the kit of parts may also be pre-dissolved, in particular if such a form of the hardening material is suitable for storage; or in the case of a thixotropic gel, the hardening material can be in a pasty or gelled form for storage/in the kit. The kit of parts may further and optionally separately include a suitable amount of a solvent (e.g. water), solvents or mixtures of solvents to mix the hardening material with, in order to obtain a pasty/liquid hardening material which can then be used in orthopedic surgery, and which will harden to a structure after being administered to a given location during orthopedic surgery.

The implant having thermoplastic properties refers to any object (e.g. implants as exemplified below) that is suitable for implantation into another object, in particular a recess in an object, e.g. in the hardening material, and having at least partial thermoplastic properties. In other words, the implant comprises a thermoplastic material which imparts thermoplastic properties such that at least a part of the implant can be liquified by energy, in particular vibrational energy applied to the implant, e.g. via a sonotrode (which may optionally be included in the kit of parts), in particular during or after the insertion of the implant into an object (e.g. a recess in the hardening material), where the implant will re-solidify due to cooling. Materials having thermoplastic properties suitable for the implant as described herein are known in the art and include, e.g., thermoplastic polymers, e.g.: resorbable or degradable polymers such as polymers based on lactic and/or glycolic acid (PL A, PLLA, PGA, PLGA etc.) or polyhydroxy alkanoates (PHA), polycaprolactone (PCL), polysaccharides, polydioxanes (PD) polyanhydrides, polypeptides or corresponding copolymers or composite materials (e.g. organic or inorganic fibers or whiskers, e.g. of calcium phosphate ceramics or glasses) containing the named polymers as a component; or non-resorbable or non-degradable polymers such as polyolefines (e.g. polyethylene), polyacrylates, polymetacrylates, polycarbonates, polyamides, polyester, polyurethanes, polysulfones, polyarylketones, polyimides, polyphenyl sulfides or liquid crystal polymers LCPs, polyacetales, halogenated polymers, in particular halogenated polyolefines, polyphenylensulfides, polysulfones, polyethers or equivalent copolymers or composite materials containing the named polymers as a component. For further exemplary thermoplasts, see, e.g., WO 2012/037699, which is incorporated by reference in its entirety, in particular page 6, line 8 to page 9, line 7.

Exemplary implants include pins and suture anchors (see, e.g., WO 2012/037699). Together with the implant, a device as disclosed herein and fasteners combined with implants having thermoplastic properties, e.g. for tenodesis, e.g. prosthetic elements, e.g. as described in WO 2011/091545, incorporated by reference in its entirety, can be used, e.g. as part of the kit of parts or the methods disclosed herein. For example, fasteners (prosthetic elements) of different shapes and forms can be used together with the implant as described herein to achieve a press-fit connection in a recess by means of the fastener (prosthetic element) and a positive-fit connection by means of the implant used together with the fastener (prosthetic element), e.g. as described in WO 2011/091545. The device is detailed further below. For example, the suture anchor may be an essentially cylindrical structure with a length of 4 mm to 20 mm, an outer diameter of 1 mm to 9 mm, and with an optional inner cavity that stretches from a proximal end into the suture anchor towards a distal end over 0.5 mm to 8 mm and having a cavity diameter of 0.2 mm to 8 mm. The inner cavity may be cylindrical or polygonal (prismatic) and/or have a tapered (conus) shape (with the largest diameter at the proximal end of the suture anchor) suitable for a sonotrode to be inserted. The pin as described herein may have the same dimensions as the anchor or have the following dimensions: length of 4 mm to 60 mm, outer diameter of 1 mm to 10 mm, optional inner cavity depth (from a proximal end into the pin towards a distal end) of 0.5 mm to 10 mm, cavity diameter 0.2 mm to 8 mm. The outer circumferential surface of the implant, e.g. suture anchor or pin, may be irregular and/or, e.g., feature protrusions, e.g. as detailed in WO 2012/037699.

It was surprisingly found that the physical properties of the hardening materials, e.g. disadvantages resulting from the brittle characteristics, porous structures and/or the limited mechanical stability of hardening materials, e.g. (hydraulic) cements, in particular CPC, can be overcome by using an implant having thermoplastic properties as described herein for insertion into the hardening material during or after hardening of the hardening material, in particular into a recess in the hardening material, in particular to create a form fit of the thermoplastic material with at least a part of the hardening material, e.g. on the surface of the material or in pores of the material. Specifically, at least a part (in particular the thermoplastic part) of the implant described herein is liquified during or after insertion into the hardening material during or after hardening of the material by transferring energy, in particular vibrational energy such as ultrasound, to the implant. The liquified part(s) of the implant can create a form fit with the hardening material, for example at locations where the hardening material would encounter stress concentration when an object is inserted into the hardening material (e.g. a screw), wherein the implant strengthens the hardening material and reduces stress. The implant can also flow into pores or cracks of the hardening material, wherein pores or cracks can refer to a “rough” surface of the hardening material where the implant material creates a form fit. These properties and mechanisms lead to a reinforcement of the hardening material's structure, a strong retention of the implant (e.g. due to the large surface area of the form fit or the large surface of implant material in pores/cracks of the hardening material into which the implant can flow) and/or a reduction of the brittleness of the hardening material.

In view of the above, the kit of parts is preferably a kit wherein the implant and hardening material (e.g. cement, e.g. CPC) are adapted relative to each other in terms of mass and size such that the implant comprises an amount of thermoplastic material that can at least partially form fit with and/or fill pores in a structure of the hardening material, i.e. during or after the hardening material has hardened at the site of application, e.g. in a recess of a hard tissue. Further details of how the implant and the hardening material interact, and how these parts are applied are provided below in the context of the methods of the invention.

The kit of parts described herein optionally further includes instructions for how the parts of the kit are used. For example, the instructions teach that the implant is for implantation into a recess in the hardening material while or followed by transmission of vibrational energy (e.g. ultrasound) to the implant such as to liquify at least a part of the implant (e.g. at least a part of the thermoplastic material of the implant). The instructions may further teach how to prepare the hardening material for filling a recess in a hard tissue (e.g. mixing it with a suitable solvent if provided as a powder or neat) and/or how to fill the hardening material in a recess in a hard tissue. The instructions may further include guidance on how to create a recess in a hard tissue suitable for filling with the hardening material and/or how to create a recess in the hardening material that was filled into the recess in a hard tissue, which recess in the hardening material would be suitable for implanting the implant. For example, the instructions may describe one or all of the method steps of the methods disclosed herein.

In an embodiment of the kit of parts according to the present invention, which may be combined with any of the embodiments of the kit of parts preaddressed or still to be addressed unless in contradiction, the kit of parts further comprises an instrument configured to administer the hardening material, in particular hydraulic cement, in particular calcium phosphate-based cement, into a recess in a hard tissue.

For example, the hardening material of the kit of parts is for at least partially filling a recess in a hard tissue. The recess can be a preexisting recess, e.g. a fracture, a damaged part of the bone, or an existing recess space, or it can be a recess that is purposively introduced into the hard tissue for treatment purposes such as attaching a ligament to a bone. The recess can also be a passage extending through the hard tissue. The kit of parts may include an instrument with which the hardening material (in its liquid or pasty form) can be introduced into a recess in a hard tissue. Suitable instruments are known in the art and include, e.g. cannulas.

In an embodiment of the kit of parts according to the present invention, which may be combined with any of the embodiments of the kit of parts preaddressed or still to be addressed unless in contradiction, the kit of parts further comprises a healing-promoting substance or a substance against inflammation, which is provided as a separate component of the kit, or which is contained in one of the discussed kit parts.

In an embodiment of the kit of parts according to the present invention, which may be combined with any of the embodiments of the kit of parts preaddressed or still to be addressed unless in contradiction, the instrument is further configured to create a recess in a structure formed by the hardening material, in particular hydraulic cement, in particular calcium phosphate-based cement, into which the implant can be implanted and/or is configured to create a recess in a hard tissue.

The instrument may also have multiple functions, for example it may be suitable to create a recess in a hard tissue (e.g. as a punching tool, a tap, a drill, or a reamer, e.g. for a recess in a bone) and/or to create a recess in the hardening material during or after hardening (e.g. as a cannula, a screw or a reamer) while also being configured to administer the hardening material to the site of application (e.g. recess in a hard tissue). The “structure formed by the hardening material” refers to the material inserted into the recess in the hard tissue, which material forms a structure that can still be pasty, partly or fully hardened. In this example, creating the recess in the structure formed by the hardening material means in particular that the instrument is kept in the pasty/liquid hardening material until a sufficient degree of setting or hardening of the hardening material is achieved such that the recess created by the volume of the instrument in the hardening material remains when the instrument is removed from the hardening material. Alternatively, the instrument can be used to create a recess in the hardened material. Whether used in pasty or hardened hardening material, the instrument may create a smooth surfaced recess, or it may create a pattern in the walls of the recess, in particular a pattern that increases the surface area of the recess, e.g. cut-outs, e.g. cut-outs with a thread form, e.g. as formed by a reamer or a screw. It is a particular advantage of the present invention that the liquified parts of the implant can create a form fit with a pattern (e.g. cut-outs or thread) in the recess in the hardened material which strengthens the pattern or thread, reduces stress concentration, and reduces the risk that the pattern/thread and the material breaks, in particular at the sites of highest stress concentration.

The instrument may comprise further functional features, such as a structural part that should be implanted in the recess, e.g. a tip of the instrument, which is separated from the instrument after the recess was created and the remaining instrument is removed, and which is subsequently fixated in the recess by the implant.

In an embodiment of the kit of parts according to the present invention, which may be combined with any of the embodiments of the kit of parts preaddressed or still to be addressed unless in contradiction, the kit of parts further comprises a tool, optionally a punching tool, a drill, a tap or a reamer, for creating a recess in a hard tissue and/or for creating a recess in a structure formed with the hardening material, in particular hydraulic cement, in particular calcium phosphate-based cement, into which structure the implant can be implanted.

In an embodiment of the kit of parts according to the present invention, which may be combined with any of the embodiments of the kit of parts preaddressed or still to be addressed unless in contradiction, the instrument is a cannula, a punching tool, a drill, a tap, a screw or a reamer.

The tap, screw or reamer can, e.g., be used to create a pattern in the walls of the recess (either in the hard tissue, the hardening material or both), in particular a pattern that increases the surface area of the recess, e.g. cut-outs, e.g. cut-outs with a thread form, e.g. as formed by a reamer or a screw. It is a particular advantage of the present invention that the liquified parts of the implant can create a form fit with a pattern (e.g. cut-outs or thread) in the recess in the hardened material which strengthens the pattern or thread, reduces stress concentration, and reduces the risk that the pattern/thread and the material breaks, in particular at the sites of highest stress concentration. For example, the screw or reamer used to create a pattern in the walls of the recess (of the had tissue, the hardening material, or both) can be configured, e.g. by suitably structured or formed threads, to improve the flow of liquified parts of the implant having thermoplastic properties in the recess.

In an embodiment of the kit of parts according to the present invention, which may be combined with any of the embodiments of the kit of parts preaddressed or still to be addressed unless in contradiction, the instrument is a screw, a tap or a reamer configured to administer the hardening material, in particular hydraulic cement, in particular calcium phosphate-based cement, into a recess in a hard tissue, and to create a recess in a structure formed by the hardening material, in particular hydraulic cement, in particular calcium phosphate-based cement, into which the implant can be implanted.

As outlined above, the instrument can be kept in the pasty/liquid hardening material (e.g. cement) until a sufficient degree of setting or hardening of the hardening material is achieved such that the recess created by the volume of the instrument in the hardening material remains when the instrument is removed from the hardening material. Thereafter, the implant can be inserted into the recess in the (hardened) hardening material.

In an embodiment of the kit of parts according to the present invention, which may be combined with any of the embodiments of the kit of parts preaddressed or still to be addressed unless in contradiction, the kit of parts further comprises reinforcement means for the hardening material, in particular hydraulic cement, in particular calcium phosphate-based cement, in particular tubular reinforcement means, in particular a porous or woven tube.

For example, reinforcement means can reinforce the hardening material structure in that the reinforcement means are arranged in the hardening material. Reinforcement means for hardening materials, e.g. cements, are known in the art and include, e.g., porous or woven structures such as tubes or dowels. For example, the reinforcement means can be positioned in the recess in the hard tissue prior to administration of the hardening material, e.g. together with the instrument used for administering the hardening material. For example, the reinforcement means do not comprise thermoplastic materials, or they do comprise thermoplastic materials, e.g. such that these materials can be welded together with the implant.

In an embodiment of the kit of parts according to the present invention, which may be combined with any of the embodiments of the kit of parts preaddressed or still to be addressed unless in contradiction, the kit further comprises a device having a body defining an inner volume, wherein
the device is suitable for receiving the implant in the inner volume, in particular the device is shaped and dimensioned to receive the implant in the inner volume,
the body of the device comprises at least one aperture,
the device is configured such that upon transmission of ultrasound energy to the implant during or after insertion of the implant into the inner volume, a liquified at least partial volume of the implant extends out of the inner volume through the at least one aperture.

The device disclosed herein can be a fastener or prosthetic element, however, it is not necessarily a fastener or prosthetic element, in particular not necessarily a fastener or prosthetic element as known in the prior art cited herein. For example, the device does not have to be dimensionally stable in itself. While fasteners and prosthetic elements are typically used to achieve a press-fit connection in a recess by means of the fastener/prosthetic element and a positive-fit connection by means of the implant used together with the fastener/prosthetic element, the device as disclosed herein is also suitable for use in the absence of press-or positive-fit connections because it can be used in the non-hardened or partially hardened hardening material, where it serves as reinforcement means and/or as a tool to liquify and direct liquified parts of the implant described herein into the non-hardened or partially hardened hardening material, which itself may not provide sufficient resistance for liquifying the implant directly.

The “body” of the device as used herein forms a partially closed structure and has at least one aperture as explained further below. For example, the body extends from a proximal end to a distal end of the body along an imaginary y-axis. The body defines an inner volume which is located inside of the body. The inner volume is suitable, i.e. shaped and sized, for receiving an implant having thermoplastic properties. In other words, the skilled person can choose/construct either the body, the implant or both relative to each other such that the implant can be received in the inner volume. The body of the instant device can be made of any dimensionally stable component(s) and have any shape. However, the body, the device and/or any components thereof may be flexible, pliable or compressible. For example, it is not a prerequisite that the body and/or the device are suitable for load bearing applications themselves(s) because the combination of the device with a liquified (and resolidified) implant results in a load bearing and load transferring structure when inserted into a hard tissue (and a hardening material).

The body comprises at least one aperture, in particular between the proximal end and the distal end. In other words, the aperture is particularly located in the “side(s)” or the “side surface(s)” of the body.

The body also, in particular in addition to the aperture, has an opening for inserting the implant into the inner volume of the body which is typically different from the aperture and does typically not coincide with the aperture. The opening is suitable for introducing the implant into the inner volume and, e.g., can be formed by a proximal section of the body, optionally by a proximal end of the body. The proximal section of the body is a section of the body along the y-axis that is closer to the proximal end than to the distal end, and lies in an imaginary proximal half of the body (that is closer to the proximal end compared to a (imaginary) distal half of the body in y-direction that is closer to the distal end of the body.). The (proximal or distal) end, on the other hand, is where the structure of the body ends.

For example, the opening for inserting the implant may, e.g., be formed by a structural element, e.g. an opening member, e.g. a collar, that is comprised in the proximal section, optionally proximal end, of the body, which opening member or collar may, e.g., form and/or shape the proximal section, optionally proximal end. The opening member or collar may also be suitably shaped to avoid that liquified parts of an implant inserted into or being inserted into the inner volume flow out of the inner volume through the opening member (i.e. in direction of the proximal end and to the outside of the inner volume via the opening), e.g. it may function as a sealing.

The device can further be configured to or it can further comprise means to cause a resistance against the implant being inserted into the inner volume along the y-axis. This resistance causes, upon ultrasound transmission, the liquification of the implant in a position of the implant being in contact with said means for causing a resistance. For example, the resistance can cause a local energy concentration and the means can be energy directors to initiate the liquification of a part of the implant. The device may itself cause the resistance, wherein components of the device serve as means to cause a resistance (e.g. the body, and/or the opening member, etc.), and/or the device may be shaped such as to cause a resistance, e.g. having a tapered or cone shape, wherein the diameter of the inner volume is reduced in direction of insertion, e.g. towards the distal end. Alternatively, or additionally, the device may comprise suitable means which cause a resistance, e.g. in the shape of protrusions reaching into the inner volume or edges (in particular edges running perpendicular to the y-axis) causing a resistance against the implant being inserted into the inner volume. For example, means can be protrusions, edges, or other 3D objects which can create a resistance against the implant, e.g. means positioned essentially perpendicular to the y-axis.

The shape of the device or the position of the means can be used to determine where at least a part of the implant liquifies. As liquification occurs, the liquified parts can extend out of the inner volume through at least one of the at least three apertures to the outside of the body (i.e. out of the inner volume). If, for example, the outside of the body facing an aperture is closed or covered by a smooth surface (e.g. the surface of a cortex), the liquified parts may flow back into the inner volume and in direction of the distal end, where, for example, the liquified parts may again extend out of the inner volume through a further aperture (or a different section of the same aperture) where, for example, a more porous structure is located such as a spongy bone into which the liquified parts can extend into and resolidify. In other words, it may, for example, not only be the structural features of the device which determine where the liquified part(s) of the implant extend out of the device, but rather, it may be a combination of the device features such as aperture location and site of implantation (what type of tissue-smooth and dense (cortex) or porous (spongy bone)-is opposite an aperture) which defines where the liquified part(s) of the implant extend out of the device.

In the following, an example of the device for implanting the implant into a hard tissue is described. In this example, the device has a body with a proximal end and a distal end spaced apart from each other along a y-axis. The ends can be rectangular, round, oval or polygonal structures. The distal end may also be a point if the body has a cone shape. The proximal section, optionally the proximal end, forms an opening (i.e. a rectangular, round, oval or polygonal structure that forms an opening) and is suitable for inserting an implant from outside the device into an inner volume that is located inside the body between the proximal end and the distal end of the body. The proximal end and the distal end are connected to each other by the body. The aperture can be located between the proximal end and distal end (i.e. in the “sides” of the body). For example, the body and/or the device may have a cage-like or net-shaped appearance where the “free space” in the sides of the cage corresponds to the aperture(s). For example, the total area of the aperture(s) (i.e. the sum of the areas of all aperture(s) in the sides of the body) can be at least equal to or greater than the total area of body surfaces enclosing the inner volume (i.e. the sum of all the areas enclosing the inner volume, i.e. closed surfaces). The distal end may be closed, i.e. delimit the interior volume, or it may be open. On the outside of the body, e.g. around the sides and/or at the distal end, further elements can be positioned. For example, a thread can extend around the body for inserting the body into a hard tissue, or the distal end can be configured as a tap, reamer or punching tip to insert the device into a hard tissue.

The dimensions of the device are chosen by the skilled person according to the needs of the specific application. For example, the dimensions are chosen relative to the hard tissue size, a depth and/or diameter of a recess in a hard tissue into which the device should be installed. For example, the device and in particular the body, has a length along the y-axis that corresponds at least to a thickness of a cortex of a bone into which the device should be inserted. Exemplary lengths of the device and/or body include 2 mm to 15 mm, 3 mm to 10 mm or 4 mm to 8 mm. Exemplary diameters or cross-sections of the device and/or body include 1.0 mm to 10 mm, 1.2 mm to 6 mm, or 1.5 to 5 mm.

For example, the at least one aperture can extend along the y-axis in at least a proximal half of the body, the at least one aperture can extend over at least 2 mm, optionally at least 4 mm in direction of the y-axis, the at least one aperture can have a width of 0.5 mm to 5 mm, optionally 1.0 mm to 4 mm and a length of 2 mm to 14 mm, optionally 3 mm to 7 mm in direction of the y-axis, a ratio of an area of the at least one aperture to a cross-section of the body can be at least 2:1, 3:1, or 4:1, or a combination thereof.

The body can have a (imaginary) proximal half in y-direction that is closer to the proximal end compared to a (imaginary) distal half of the body in y-direction that is closer to the distal end of the body. In an example, the at least one aperture is located in the proximal half of the body and optionally extends (e.g. along the y-axis) from the proximal half of the body into the distal half of the body. The cross-section of the body is the cross-section perpendicular to the y-axis, and refers to the widest cross-section of the body.

The device can have any geometrical shape including a cylindrical, cube, cuboid, cone, pyramid or prism shape, wherein the prism can be polygonal. The device can be made of titanium, steel, zinc, magnesium, alloys thereof, calcium phosphate ceramics, in particular hydroxyapatite, high-performance plastics, in particular polyether ether ketone (PEEK), polyetherketoneketone (PEKK), polyaryletherketone (PAEK), polysulfones (PSU) or polyphenylene sulfides (PPS).

In an embodiment of the kit of parts according to the present invention, which may be combined with any of the embodiments of the kit of parts preaddressed or still to be addressed unless in contradiction,
the body extends from a proximal end to a distal end along a y-axis,
a proximal section of the body, optional a proximal end of the body, forms an opening for inserting the implant into the inner volume, and
the at least one aperture is positioned between the opening and the distal end of the body.

In an embodiment of the kit of parts according to the present invention, which may be combined with any of the embodiments of the kit of parts preaddressed or still to be addressed unless in contradiction, at least one of:
the proximal section of the body is or comprises a collar,
the body is a net-shaped body,
the body is compressible, in particular a cross-section of the body is compressible, in particular perpendicular to the y-axis, in particular wherein the at least one aperture is configured to allow for a compression of the body, in particular of the cross-section of the body, in particular perpendicular to the y-axis.
or a combination thereof.

For the example of a net-shaped body, the aperture(s) can be one or more of the voids in the net. For example, the body can be compressible.

In an embodiment of the kit of parts according to the present invention, which may be combined with any of the embodiments of the kit of parts preaddressed or still to be addressed unless in contradiction, the implant is configured to be at least partially liquified by ultrasound, and optionally wherein the kit of parts further comprises a sonotrode.

As outlined above, it is preferred that the implant comprises an amount of thermoplastic material that can create a form fit with at least a part of the hardening material (e.g. cement) in the recess and/or at least partially fill pores in a structure of the hardening material (e.g. cement), in particular after the hardening material has hardened at the site of application, e.g. in a recess of a hard tissue. In addition to the form fit, the liquified part(s) of the implant can fill part of or the entire recess in the hardening material (e.g. cement). The implant can be chosen such that there is sufficient thermoplastic material for this purpose; and/or the recess in the hardening material can be created with dimensions that allow for the partial or complete form fit and/or filling of the pores and/or the recess with the thermoplastic material of the implant. For this purpose, the skilled person in the field of orthopedic implants understands that the implant is at least partially liquified and that it is the liquified part(s) of the implant which flow over the hardening material and/or into the pores and recess where these parts resolidify to achieve the described form fit and/or filling of the pores and/or recess with the thermoplastic material. Suitable sonotrodes for use in the kit of parts (and the below-described methods) are known in the art and can be routinely chosen by the skilled person, e.g. sonotrodes which transfer ultrasonic vibrations with a frequency between 15 and 30 kHz. The sonotrodes for use in the present invention are configured such that they can be used for transmitting ultrasound to the implant and, thereby, at least partially liquifying the implant during or after implantation, in particular under standard implantation conditions, for example at a body temperature of the subject of surgery.

In an embodiment of the kit of parts according to the present invention, which may be combined with any of the embodiments of the kit of parts preaddressed or still to be addressed unless in contradiction, the implant is a pin, an implant for tenodesis or a suture anchor, optionally wherein the kit further comprises a suture for use with the suture anchor.

As noted above, exemplary implants include pins, suture anchors (see, e.g., WO 2012/037699, incorporated by reference in its entirety), and can be used together with a device disclosed herein and/or fasteners (prosthetic elements) combined with implants having thermoplastic properties, e.g. for tenodesis, e.g. as described in WO 2011/091545, incorporated by reference in its entirety. Also, further fastening devices (prosthetic elements) for use together with the present implant are disclosed in U.S. Pat. Nos. 7,335,205, 7,008,226, US 2006/0105295, US 2009/131947, WO 2009/132472, WO 2008/34276, WO 2010/127462 and WO 2010/045751, all incorporated by reference in their entireties. Essentially, the systems and implants mentioned above can be used in the present invention such that the recess into which they are inserted corresponds at least partially to the recess in the hardening material (e.g. cement, e.g. instead of the recess “directly” in the hard tissue) and optionally partially to the recess in the hard tissue. Exemplary indications include:
joint implants, in particular for extremities such as fingers or toes, e.g. as disclosed in WO 2004/017857 A1, incorporated by reference in its entirety;
arthrodesis systems to bridge a joint or osteotomies, e.g. by reinforcing the sites to be bridged with the hardening material;
intervertebral spacers or osteotomic wegdes, e.g. as disclosed in US 2010/0274358 A1, incorporated by reference in its entirety;
a use of the kit of parts together with a cannulated screw or pin-like anchoring device as disclosed in WO 2011/054124 A1, incorporated by reference in its entirety, wherein, e.g. the hardening material is administered to a recess in a hard tissue through the bore of the device (e.g. while also applying ultrasound to support the flow of the material, in particular if the material is thixotropic), and subsequently the implant having thermoplastic properties is inserted into the pedicle anchor device where, upon ultrasound transmission, it liquifies and also exits through the bore to interact with the hardening material already in place to create the aforementioned form fit and/or filling of pores which strengthens the structure and increases the load bearing properties of the hardening material; and/or
dental implants, e.g. as disclosed in U.S. Pat. No. 8,357,201 B2, CH 712 758 A2, and CH 712 757 A2, all incorporated by reference in their entireties.

In a further aspect, which may be combined with any of the embodiments and aspects preaddressed or still to be addressed unless in contradiction, the present invention is directed to a method for implanting an implant into a hard tissue comprising the steps, optionally in the given order:
(a1) providing a hard tissue with a recess that is at least partially filled with a hardened hardening material for orthopedic use, in particular hydraulic cement, in particular a calcium phosphate-based cement, as defined herein, wherein the hardened hardening material, in particular hydraulic cement, in particular calcium phosphate-based cement, comprises a recess, and inserting an implant having thermoplastic properties as defined herein into the recess in the hardening material, in particular hydraulic cement, in particular calcium phosphate-based cement, while or followed by transmitting ultrasound to the implant for a time sufficient to liquify the implant to the extent that liquification results in fixation of the implant in the recess in the hardening material, in particular hydraulic cement, in particular calcium phosphate-based cement.

In a further aspect, which may be combined with any of the embodiments and aspects preaddressed or still to be addressed unless in contradiction, the present invention is directed to a method for implanting an implant into a hard tissue comprising the steps, optionally in the given order:
(a2) providing a hard tissue with a recess that is at least partially filled with a hardened hardening material for orthopedic use, in particular hydraulic cement, in particular calcium phosphate-based cement, as defined herein, and creating, optionally drilling, a recess into the hardened hardening material, in particular hydraulic cement, in particular calcium phosphate-based cement, and
inserting an implant having thermoplastic properties as defined herein into the recess in the hardening material, in particular hydraulic cement, in particular calcium phosphate-based cement, while or followed by transmitting ultrasound to the implant for a time sufficient to liquify the implant to the extent that liquification results in fixation of the implant in the recess in the hardening material, in particular hydraulic cement, in particular calcium phosphate-based cement.

In a further aspect, which may be combined with any of the embodiments and aspects preaddressed or still to be addressed unless in contradiction, the present invention is directed to a method for implanting an implant into a hard tissue comprising the steps, optionally in the given order:
(a3) providing a hard tissue with a recess that is at least partially filled with a non-hardened or partially hardened hardening material for orthopedic use, in particular hydraulic cement, in particular a calcium phosphate-based cement, as defined herein, followed by providing a device (e.g. as described herein) having a body defining an inner volume and comprising at least one aperture, inserting the device into the hardening material, and inserting an implant having thermoplastic properties through an opening of the device into the inner volume of the device while or followed by transmitting ultrasound to the implant to liquify the implant to the extent that a liquified at least partial volume of the implant extends out of the inner volume of the device through the at least one aperture, wherein the insertion of the device into the hardening material and the insertion of the implant into the device is carried out while the hardening material is non-hardened or partially hardened.

The hard tissue in the present methods is, for example, a bone, in particular a bone with low bone density or an osteoporotic bone which itself would provide little or insufficient stability for fixation of a part (e.g. a ligament or an implant structure) thereto. The recess in the hard tissue can be a preexisting recess, e.g. a fracture, a damaged part of the bone, or an existing recess space, or it can be a recess that is created in the hard tissue in the context of the present methods by suitable and known orthopedic techniques. Typically, and for example, the recess in the hard tissue reaches through the cortical bone, in particular into the spongy bone (trabecular bone). The dimensions of the recess are typically adapted to the requirements of the orthopedic actions, or are those dimensions already present, e.g., in the case of a fracture. The recess, in one method aspect, can be already filled with a hardening material (e.g. cement) as described herein, e.g. in the context of a step preceding the method. The amount of hardening material that is filled into the recess in the hard tissue is routinely chosen by the skilled person, e.g. the surgeon depending on the needs for the orthopedic action, the bone size, the recess size and/or the porosity of the bone material around the recess.

The term “hardened hardening material” as used herein means that the material is hardened at least to the extent that it exhibits load bearing and/or load transferring properties, e.g. that the hardening material has become a solid.

The term “non-hardened hardening material” means that the hardening reaction of the material has not begun or only begun to a non-measurable extent (e.g. no change in viscosity can be measured as defined herein), the non-hardened hardening material can, e.g., be liquid and/or not dimensionally stable. Suitable material properties including viscosity for use in surgery, e.g. for injection into a hard tissue recess, are known to the skilled person. For example, the non-hardened hardening material can have a viscosity that is particularly suitable for injection, e.g. with a syringe, into a recess, in particular for injection through a syringe with less than 38 N, less than 30 N, less than 15 N or less than 12 N. For example, the non-hardened hardening material can have a viscosity of less than 500 Pas measured at 20° C., optionally a viscosity between 10 Pas and 300 Pas or between 50 Pas and 150 Pas at 20° C.

All viscosities disclosed herein refer to viscosities at 20° C. and as measured by, for example, a controlled shear stress-shear rate rheometer (see, e.g., M. Nehdi, S. Al Martini, Cement and Concrete Research, Volume 39, Issue 11, 2009, Pages 1007-1016).

The term “partially hardened hardening material” means that the hardening reaction of the material may have begun, for example locally, but that the material is still pasty, viscous and not solid, e.g. to the extent that it can be displaced without destruction and/or by manual force by a surgeon without destruction, e.g. having a viscosity between 200 Pas and 1000 Pas measured at 20° C.

The term “non-hardened or partially hardened hardening material” therefore, for example, refers to the hardening material having a viscosity between, e.g., 10 Pas and 1000 Pas at 20° C.

It is preferred to insert the device into the hardening material and/or the implant into the device when the hardening material is non-hardened or partially hardened, i.e. is liquid or pasty, e.g. having a viscosity between 50 Pas and 150 Pas at 20° C.

The hardening material (e.g. cement) may already comprise a recess, or a recess may be created in the hardening material in the context of the present method. For example, the recess can be created in the hardening material by means of the instrument that was used for filling the hardening material into the recess in the hard tissue, or it can be formed after filling and optionally drying, e.g. with a punching tool, a screw, a drill, a tap or a reamer. The dimensions of the recess in the hardening material can be chosen, e.g., depending on the size of the implant, the object(s) that are implanted together with the implant into the recess and the overall size of the bone and the volume of the hardening material.

In an example, the implant can be a pin or a suture anchor and the recess can, e.g., have
a cross-section corresponding to the cross-section of the pin or suture anchor, or
a cross-section that is smaller than the cross-section of the pin or suture anchor to the extent that an insertion of the pin or suture anchor is possible, or
a cross-section corresponding to or being larger than the cross-section of the pin or suture anchor and has a depth that is shorter than the length of the at least one pin or suture anchor.

For example, if a tissue and/or a corresponding prosthetic element is to be implanted and fixated in the recess of the hardening material (e.g. cement), the dimensions of the recess are chosen such that the tissue and/or the element can fit, optionally by a press-fit, in the recess, whereupon the implant is used to fixate the element in the recess by flowing into the hardening material upon liquification. In other words, in this example, the recess cross-section would be larger than the cross-section of the implant and/or its depth would be longer than the depth of the implant, wherein the cross-section and depth are suitable for a press-fit of, e.g. a tissue (e.g. a ligament) and/or a prosthetic element such as, e.g. described in WO 2011/091545, being incorporated by reference in its entirety.

The step of inserting an implant having thermoplastic properties as defined herein, and for all methods disclosed herein, into the recess in the hardening material (e.g. cement) may be preceded or accompanied by inserting further components to be implanted and fixated into the recess in the hardening material, e.g. as described above in the context of a tissue, reinforcement means and/or a corresponding prosthetic element and/or the device as disclosed herein. The expression “inserting the implant into the recess” is to be understood as positioning the implant at least such that it is spatially at least partially located within the recess in the hardening material (e.g. cement; and/or the recess in the hard tissue) and can flow at least partially into the hardening material and/or hard tissue structure. The expression includes the case wherein the implant is inserted into the recess while making physical contact with the recess structure (i.e. the hardening material), and the case wherein the implant is inserted into an element that is previously or simultaneously positioned in the recess, such as a prosthetic element or device, from or through which element the liquified implant can at least partially flow into the recess to achieve a form fit with and/or flow into the hardening material and/or bone structure (pores). The term “hardening material and/or bone structure” is used because, in the case of spongy bone material, the hardening material and the bone material may be “mixed” and both materials may come into contact with the liquified implant due to the materials' porous nature—this applies to all embodiments disclosed herein and is not limited to prosthetic elements or the device. For example, the further elements mentioned above may be inserted into the recess to create a press-fit of the elements in the recess and subsequently the implant is inserted into prosthetic element (i.e. globally into the recess) where it can be liquified and flow out from a suitable opening in the prosthetic element and onto and/or into the hardening material for fixating the prosthetic element and thereby a further element such as a ligament (see, e.g., WO 2011/091545 for exemplary prosthetic element and implant combinations).

As detailed above, the device disclosed herein can be a fastener or prosthetic element, however, it is not necessarily a fastener or prosthetic element, in particular not necessarily a fastener or prosthetic element as known in the prior art cited herein. For example, the device does not have to be dimensionally stable in itself. While fasteners and prosthetic elements are typically used to achieve a press-fit connection in a recess by means of the fastener/prosthetic element and a positive-fit connection by means of the implant used together with the fastener/prosthetic element, the device as disclosed herein is also suitable for use in the absence of press-or positive-fit connections because it can be used in the non-hardened or partially hardened hardening material, where it serves as reinforcement means and/or as a tool to liquify and direct liquified parts of the implant described herein into the non-hardened or partially hardened hardening material, which itself may not provide sufficient resistance for liquifying the implant directly. As described herein, transmitting ultrasound to the implant during or after inserting the implant into the device to liquify the implant to the extent that a liquified at least partial volume of the implant extends out of the inner volume of the device through the at least one aperture, can also typically, and for example, mean that the liquified at least partial volume of the implant extends out of the device and into the non-hardened or partially hardened hardening material, e.g. displacing a volume thereof, and resolidifying in the non-hardened or partially hardened hardening material, e.g. followed by the hardening of the hardening material. For example, the liquified parts of the implant can displace the non-hardened or partially hardened hardening material while forming, e.g., roundish and/or soft-edged shapes of liquified and subsequently re-solidified implant material which aids in retaining the implant and reduce stress due to the roundish shape.

As noted above, the implant has at least partial thermoplastic properties. In other words, the implant comprises a thermoplastic material which imparts thermoplastic properties such that at least a part of the implant can be liquified by the vibrational energy applied to the implant, e.g. via a sonotrode. The energy transfer and liquification of the implant in the context of the present methods can be done during its insertion, or subsequent to its insertion depending on the application and the skilled person can routinely chose the suitable moment and means for energy transfer and liquification. The liquified (parts of the) implant can flow onto and/or into the hardening material structure and resolidify thereon/therein, which leads to a fixation of the implant.

In a further aspect, which may be combined with any of the embodiments and aspects preaddressed or still to be addressed unless in contradiction, the present invention is directed to a method for implanting an implant into a hard tissue comprising the steps, optionally in the given order:
(a) providing a kit of parts as described herein, or providing the components defined in the kit of parts as described herein individually,
(b1) providing a hard tissue with a recess, or
(b2) providing a hard tissue and creating, optionally drilling, a recess into the hard tissue,
(c) filling the recess at least partially with a hardening material for orthopedic use (e.g. as described herein), in particular hydraulic cement, in particular calcium phosphate-based cement, and hardening the hardening material,
(d1) creating, optionally drilling, a recess into the hardening material, in particular hydraulic cement, in particular calcium phosphate-based cement, followed by inserting the implant into the recess in the hardening material, in particular hydraulic cement, in particular calcium phosphate-based cement, while or followed by transmitting ultrasound to the implant for a time sufficient to liquify the implant to the extent that liquification results in fixation of the implant in the recess in the hardening material, in particular hydraulic cement, in particular calcium phosphate-based cement.

In a further aspect, which may be combined with any of the embodiments and aspects preaddressed or still to be addressed unless in contradiction, the present invention is directed to a method for implanting an implant into a hard tissue comprising the steps, optionally in the given order:
(a) providing a kit of parts as described herein, or providing the components defined in the kit of parts as described herein individually,
(b1) providing a hard tissue with a recess, or
(b2) providing a hard tissue and creating, optionally drilling, a recess into the hard tissue,
(c) filling the recess at least partially with a hardening material for orthopedic use (e.g. as described herein), in particular hydraulic cement, in particular calcium phosphate-based cement,
(d2) inserting the implant into the non-hardened or partially hardened hardening material while or followed by transmitting ultrasound to the implant for a time sufficient to liquify the implant to the extent that liquification results in fixation of the implant in the hardening material and hardening the hardening material.

In a further aspect, which may be combined with any of the embodiments and aspects preaddressed or still to be addressed unless in contradiction, the present invention is directed to a method for implanting an implant into a hard tissue comprising the steps, optionally in the given order:
(a) providing a kit of parts as described herein, or providing the components defined in the kit of parts as described herein individually,
(b1) providing a hard tissue with a recess, or
(b2) providing a hard tissue and creating, optionally drilling, a recess into the hard tissue,
(c) filling the recess at least partially with a hardening material for orthopedic use (e.g. as described herein), in particular hydraulic cement, in particular calcium phosphate-based cement,
(d3) providing a device having a body defining an inner volume and comprising at least one aperture,
inserting the device into the hardening material,
and inserting the implant through an opening of the device into the inner volume of the device while or followed by transmitting ultrasound to the implant to liquify the implant to the extent that a liquified at least partial volume of the implant extends out of the inner volume of the device through the at least one aperture,
wherein the insertion of the device into the hardening material and the insertion of the implant into the device is carried out while the hardening material is non-hardened or partially hardened.

The kit of parts for use in the present methods is the kit of parts disclosed herein and all definitions provided in the context of the kit of parts apply for its use in the instant methods. The components of the kit of parts can be provided individually, i.e. not as a kit of parts, for use in the present method and the definitions provided for these parts in the context of the kit apply.

The hard tissue of all methods disclosed herein can be a hard tissue as explained above with a preexisting recess (step (b1)), or without a recess which is then created (e.g. during step (b2)) during the method, e.g. by known methods in the art including, e.g. (antegrade or retrograde) drilling with a drill, by creating the recess by punching, or by creatin the recess with a tap or a reamer (optionally after drilling or punching). The recess in the hard tissue may also be created simultaneously with filling the hardening material (e.g. cement), for example with an instrument that is suitable for creating a recess and for administering hardening material (e.g. cement), e.g. while the recess is created. Such an instrument can be, e.g. a drill, a punching tool, a tap or a reamer that includes a suitable channel for administering hardening material.

The dimensions of the recess can be as described above for all methods described herein.

The step of filling hardening material (e.g. cement) may require that the hardening material is first hydrated or mixed with a suitable solvent, e.g. if the hardening material is provided in a storable form such as a powder or dry (neat) substance. This hydration or mixing with solvent can be done with a solvent that is provided in the kit of parts or with other solvent(s) provided individually. The skilled person can either follow instructions (e.g. provided in the kit of parts or provided with the hardening material (e.g. cement) powder) or his knowledge in the art to achieve a suitable hydrated or dissolved hardening material for filling a recess. In any case, the skilled person is aware that the step of filling the hardening material relates to a hardening material in a form (e.g. hydrated to a pasty or liquid form) that is suitable for further processing. In the case that a thixotropic material is used, the skilled person can additionally apply ultrasound to the material while filling such as to achieve a more liquid state of the material during the filling.

In one example, after filling the hardening material (e.g. cement), the hardening material is hardened at least to the extent that it is sufficiently dimensionally stable for the step of inserting the implant and optionally further elements as described above and below into a recess in the hardening material. In an alternative example, the implant and optionally further elements can be inserted into hardening material that is still non- or partially hardened, e.g. pasty, which can result in a mixing of the thermoplastic implant material and the hardening material. This example is particularly applicable if the implant is inserted into a further element (e.g. a device as disclosed herein or a prosthetic element) in the recess out of which (e.g. via the aperture described for the device, or an opening or bore in a prosthetic element) the liquified part(s) of the implant flow out into the recess in the hardening material or hard tissue. As outlined above, the recess in the hardening material can be introduced after or during hardening by suitable instruments.

In the step wherein the implant is liquified to the extent that liquification results in fixation of the implant in the recess in the hardening material, the liquified part(s) of the implant can create a form fit with the hardening material, for example at locations where the hardening material would encounter stress concentration when an object is inserted into the hardening material (e.g. a screw), wherein the implant strengthens the hardening material and reduces stress. The implant can also flow into pores or cracks of the hardening material, wherein pores or cracks can refer to a “rough” surface of the hardening material where the implant material creates a form fit. These properties and mechanisms lead to a reinforcement of the hardening material's structure, a strong retention of the implant (e.g. due to the large surface area of the form fit or the large surface of implant material in pores/cracks of the hardening material into which the implant can flow) and/or a reduction of the brittleness of the hardening material.

As described above, transmitting ultrasound to the implant during or after inserting the implant into the device to liquify the implant to the extent that a liquified at least partial volume of the implant extends out of the inner volume of the device through the at least one aperture, can also typically, and for example, mean that the liquified at least partial volume of the implant extends out of the device and into the non-hardened or partially hardened hardening material, e.g. displacing a volume thereof, and resolidifying in the non-hardened or partially hardened hardening material, e.g. followed by the hardening of the hardening material. For example, the liquified parts of the implant can displace the non-hardened or partially hardened hardening material while forming, e.g., roundish and/or soft-edged shapes of liquified and subsequently re-solidified implant material which aid in retaining the implant and reduce stress due to the roundish shape.

The recess in the hardening material and/or in the hard tissue can be made with an instrument that creates a pattern, e.g. cut-outs or a thread, in the recess (in the walls thereof), e.g. with a tap, screw or reamer can, in particular a pattern that increases the surface area of the recess. It is a particular advantage of the present invention that the liquified parts of the implant can create a form fit with a pattern (e.g. cut-outs or thread) in the recess in the hardened material which strengthens the pattern or thread, reduces stress concentration, and reduces the risk that the pattern/thread and the material breaks, in particular at the sites of highest stress concentration.

Also disclosed is a method for implanting an implant into a hard tissue comprising the steps, optionally in the following order:
(a) providing a kit of parts as described herein, or providing the components defined in the kit of parts as described herein individually,
(b1) providing a hard tissue with a recess, or
(b2) providing a hard tissue and creating, optionally drilling, a recess into the hard tissue,
(c) filling the recess at least partially with the hardening material, in particular hydraulic cement, in particular calcium phosphate-based cement, and creating a recess into the hardening material, in particular hydraulic cement, in particular calcium phosphate-based cement,
(d) hardening the hardening material, in particular cement,
(e) inserting the implant into the recess in the hardening material, in particular hydraulic cement, in particular calcium phosphate-based cement, while or followed by transmitting ultrasound to the implant for a time sufficient to liquify the implant to the extent that liquification results in fixation of the implant in the recess in the hardening material, in particular hydraulic cement, in particular calcium phosphate-based cement.

As outlined above, in one method described herein, the recess can be created in the hardening material (e.g. cement) by means of the instrument that was used for filling the hardening material into the recess in the hard tissue, or it can be formed after filling and optionally drying, e.g. with a punching tool, a drill, a tap or a reamer. For example, the hardening material can be filled into the recess in the hard tissue with an instrument that is suitable for creating a recess in the hardening material and for administering hardening material (and optionally for creating a pattern in the recess as described above). Such an instrument can be, e.g. a cannula, a drill, a punching tool, a tap or a reamer that includes a suitable channel for administering hardening material. Alternatively, a suitable instrument (e.g. a cannula, a screw, a drill, a punching tool, a tap or a reamer) can be inserted into the still pasty (not fully hardened) hardening material in order to create a recess (e.g. with a pattern) in the hardening material, and the instrument can be removed from the hardening material once it is sufficiently dimensionally stable such that the recess (and optionally the pattern) created by the instrument remains in the hardening material after removal of the instrument therefrom.

Also disclosed is a method for implanting an implant into a hard tissue comprising the steps:
(a) providing a kit of parts as described herein, or providing the components defined in the kit of parts disclosed herein individually,
(b1) providing a hard tissue with a recess, or
(b2) providing a hard tissue and creating, optionally drilling, a recess into the hard tissue,
(c) filling the recess at least partially with the hardening material, in particular hydraulic cement, in particular calcium phosphate-based cement,
(d) inserting the implant into the non-hardened hardening material, in particular hydraulic cement, in particular calcium phosphate-based cement, while or followed by transmitting ultrasound to the implant for a time sufficient to liquify the implant to the extent that liquification results in fixation of the implant in the hardening material, in particular hydraulic cement, in particular calcium phosphate-based cement, and
(e) hardening the hardening material.

In the above aspect, the implant and optionally further elements can be inserted into hardening material that is still pasty and not yet (fully) hardened. “Non-hardened” as used herein includes the pasty, not yet (fully) hardened state of the material in which the material is not yet load bearing and/or still allows for a thermoplastic implant or a further element (e.g. a prosthetic element or device) to be “pushed” into the material. Upon transfer of ultrasound to the implant a mixing of thermoplastic implant material and hardening material can occur. This example is particularly applicable if the implant is inserted into a further element (e.g. a prosthetic element or device) inserted into the non-hardened material, out of which (e.g. via an the aperture of the device or an opening or bore in the prosthetic element) the liquified part(s) of the implant flow out and contact and/or mix with the hardening material and/or hard tissue.

In a further aspect, which may be combined with any of the embodiments and aspects preaddressed or still to be addressed unless in contradiction, the present invention is directed to a method for implanting an implant into a hard tissue comprising the steps:
(a) providing a kit of parts as defined herein, or providing the components defined in the kit of parts disclosed herein individually,
(b1) providing a hard tissue with a recess, or
(b2) providing a hard tissue and creating, optionally drilling, a recess into the hard tissue,
(c) filling the recess at least partially with the hardening material, in particular hydraulic cement, in particular calcium phosphate-based cement, and creating a recess into the hardening material, in particular hydraulic cement, in particular calcium phosphate-based cement, before or after hardening of the hardening material, in particular hydraulic cement, in particular calcium phosphate-based cement, wherein the recess in the hardening material has a proximal side facing the outside of the hard tissue and a distal side facing the interior of the hard tissue,
(d) optionally hardening the hardening material, in particular hydraulic cement, in particular calcium phosphate-based cement, and
guiding the implant through the recess in the hardening material, in particular hydraulic cement, in particular calcium phosphate-based cement, while or followed by transmitting ultrasound to the implant for a time sufficient to liquify the implant to the extent that liquification results in fixation of the implant in an area of the hard tissue adjacent to the distal end of the recess and/or in the recess in the hardening material, in particular hydraulic cement, in particular calcium phosphate-based cement, or
guiding the device as disclosed herein through the recess in the hardening material, in particular hydraulic cement, in particular calcium phosphate-based cement, and inserting the implant into the device when the hardening material is non-hardened or partially hardened, while or followed by transmitting ultrasound to the implant for a time sufficient to liquify the implant to the extent that liquification results in fixation of the implant in an area of the hard tissue adjacent to the distal end of the recess and/or in the recess in the hardening material, in particular hydraulic cement, in particular calcium phosphate-based cement.

In the above embodiment, the hardening material is used to stabilize an area of the hard tissue and the implant is positioned and fixated “behind” the hardening material as seen from the outside of the hard tissue to the inside of the hard tissue. The implant can still create a form fit with the hardening material and/or flow into pores/cracks, however, it is positioned similar to a plug behind the hardening material, and at least not entirely but optionally additionally in the recess in the hardening material. For this aspect, the implant is preferably an anchor, e.g. a suture anchor.

In an embodiment of the methods according to the present invention, which may be combined with any of the embodiments of the methods preaddressed or still to be addressed unless in contradiction, the recess in the hardening material, in particular hydraulic cement, in particular calcium phosphate-based cement, of the instant methods is created by inserting an instrument, optionally a cannula, a screw or a reamer, into the non-hardened or partially hardened hardening material, in particular hydraulic cement, in particular calcium phosphate-based cement, in the recess of the hard tissue before hardening the hardening material.

An instrument can be inserted into the non-hardened or partially hardened hardening material to create a recess and, for example, be removed as soon as the hardening material is sufficiently dimensionally stable such that the recess created by the instrument remains after its removal.

In an embodiment of the methods according to the present invention, which may be combined with any of the embodiments of the methods preaddressed or still to be addressed unless in contradiction, the recess in the hardening material, in particular hydraulic cement, in particular calcium phosphate-based cement, of the instant methods is created by an instrument into the non-hardened or partially hardened hardening material in the recess of the hard tissue, optionally a cannula, a screw or a reamer, wherein the instrument is further used for at least partially filling the recess in the hard tissue with the hardening material, in particular hydraulic cement, in particular calcium phosphate-based cement.

As outlined above, the instrument may have a suitable channel through which the hardening material (e.g. cement) can be filled into the recess in the hard tissue.

For example, the instrument can be removed from the hardening material, in particular hydraulic cement, in particular calcium phosphate-based cement, in the recess in the hard tissue after hardening of the hardening material, in particular hydraulic cement, in particular calcium phosphate-based cement.

In this context of instrument removal, “after hardening” means the hardening process has progressed to the extent that the hardening material is sufficiently dimensionally stable such that the recess and/or a pattern in the walls of the recess created by the instrument remains after its removal.

In an embodiment of the methods according to the present invention, which may be combined with any of the embodiments of the methods preaddressed or still to be addressed unless in contradiction, the creating of the recess in the hard tissue, the filling of the recess with the hardening material, in particular hydraulic cement, in particular calcium phosphate-based cement, and/or the creating of the recess in the hardening material, in particular hydraulic cement, in particular calcium phosphate-based cement, is performed with the same instrument, optionally a cannula, a screw or a reamer.

The tap, screw or reamer can, e.g., be used to create a pattern in the walls of the recess (either in the hard tissue, the hardening material or both), in particular a pattern that increases the surface area of the recess, e.g. cut-outs, e.g. cut-outs with a thread form, e.g. as formed by a reamer or a screw.

For example the instrument can be a cannula, a screw or a reamer configured to administer the hardening material, in particular hydraulic cement, in particular calcium phosphate-based cement, into the recess in the hard tissue.

In an embodiment of the methods according to the present invention, which may be combined with any of the embodiments of the methods preaddressed or still to be addressed unless in contradiction, at least one of the implant, the recess in the hardening material, in particular hydraulic cement, in particular calcium phosphate-based cement, or both is/are configured such that at least one of: the liquified part of the implant makes a form fit with at least a part of the hardening material, in particular hydraulic cement, in particular calcium phosphate-based cement, the liquified part of the implant fills pores in the recess in the hardening material, in particular hydraulic cement, in particular calcium phosphate-based cement, the liquified part of the implant fills the entire recess in the hardening material, in particular hydraulic cement, in particular calcium phosphate-based cement, or a combination thereof, when inserting the implant into the recess in the hardening material, in particular hydraulic cement, in particular calcium phosphate-based cement, while or followed by transmitting ultrasound to the implant for a time sufficient to liquify the implant to the extent that liquification results in fixation of the implant in the recess in the hardening material, in particular hydraulic cement, in particular calcium phosphate-based cement.

In this example, the liquified part(s) of the implant flow onto a surface of and/or into the hardening material (e.g. cement, and/or optionally bone) to create a form fit with at least a part of the hardening material and/or to positively fill in the voids (hollow spaces, e.g. cracks or pores) in the hardening material (and optionally bone) where these liquified parts resolidify to achieve the described form fit and/or filling of the pores and cracks with the thermoplastic material. The liquified parts may also fill or form fit with further orthopedic elements inserted into the recess in the hardening material and/or the recess in the hardening material. This results not only in improved fixation of the implant (and further elements) in the recess due to the large surface area into which the thermoplastic material is distributed, but also in strengthening the hardening material structure because the thermoplastic material reduces stress concentration and/or fills pores and cracks.

In the aspect in which fixation of the implant is achieved in an area of the hard tissue adjacent to the distal end of the recess in the hardening material, the implant may optionally also be located in the recess in addition to being located “behind” the hardening material when viewed from outside to the inside of the hard tissue.

In an embodiment of the methods according to the present invention, which may be combined with any of the embodiments of the methods preaddressed or still to be addressed unless in contradiction, the implant is an implant for tenodesis or a suture anchor that comprises a suture threaded through a suture conduit and optionally wherein the method further comprises threading the suture through the suture conduit of the suture anchor.

In an embodiment of the methods according to the present invention, which may be combined with any of the embodiments of the methods preaddressed or still to be addressed unless in contradiction, the method further comprises attaching a tissue, optionally a ligament, with the suture of the suture anchor to the hard tissue, optionally after insertion of the implant into the recess in the hardening material, in particular hydraulic cement, in particular calcium phosphate-based cement.

In an embodiment of the methods according to the present invention, which may be combined with any of the embodiments of the methods preaddressed or still to be addressed unless in contradiction, the instrument for at least partially filling the recess in the hard tissue with the hardening material, in particular hydraulic cement, in particular calcium phosphate-based cement, and/or creating the recess in the hardening material, in particular hydraulic cement, in particular calcium phosphate-based cement, comprises an object to be implanted, e.g. at the tip of the instrument, wherein during the filling and/or creating of the recess, the object is implanted and the instrument is subsequently removed.

For example, the object to be implanted can be an object for inserting and/or retaining a suture, a basket structure or any other object that should be implanted and subsequently fixated by the implant in the instant method.

In an embodiment of the methods according to the present invention, which may be combined with any of the embodiments of the methods preaddressed or still to be addressed unless in contradiction, the method further comprises before the step of filling the recess in the hard tissue with the hardening material, in particular hydraulic cement, in particular calcium phosphate-based cement, the step of inserting reinforcement means for the hardening material, in particular tubular reinforcement means, in particular a porous or woven tube, followed by filling the recess comprising the reinforcement means with the hardening material, in particular hydraulic cement, in particular calcium phosphate-based cement.

For example, reinforcement means can be positioned in the recess in the hard tissue such that the hardening material prior to administration of the hardening material encloses and optionally flows into the reinforcement means. For example, the reinforcement means can be inserted into the recess together with and in the same step as the instrument for administering the hardening material. For example, the reinforcement means do not comprise thermoplastic materials, or they do comprise thermoplastic materials, e.g. such that these materials can be welded together with the implant when the implant is inserted into the recess in the hardening material and ultrasound is applied to the implant.

In an embodiment of the methods according to the present invention, which may be combined with any of the embodiments of the methods preaddressed or still to be addressed unless in contradiction, the method further comprises at least one of:
inserting a tissue, optionally a ligament, into the recess in the hardening material, in particular hydraulic cement, in particular calcium phosphate-based cement, before or during insertion of the implant into the recess in the hardening material, in particular hydraulic cement, in particular calcium phosphate-based cement;
inserting a further prosthetic element into the recess in the hardening material, in particular hydraulic cement, in particular calcium phosphate-based cement, before or during insertion of the implant into the recess in the hardening material, in particular hydraulic cement, in particular calcium phosphate-based cement, optionally a prosthetic element that has a press-fit in the recess; or
a combination thereof.

Examples for prosthetic elements and tissue fixation are provided in WO 2011/091545 incorporated by reference in its entirety.

In an embodiment of the methods according to the present invention, which may be combined with any of the embodiments of the methods preaddressed or still to be addressed unless in contradiction, at least one of:
the proximal section of the body of the device is or comprises a collar,
the body of the device is a net-shaped body,
the body of the device is compressible, in particular a cross-section of the body, in particular perpendicular to the y-axis, in particular wherein the at least one aperture is configured to allow for a compression of the body, in particular of the cross-section of the body, in particular perpendicular to the y-axis.
or a combination thereof.

For example, if the body of the device is a net-shaped body, the aperture can be one of the voids in the net.

In an embodiment of the kit of parts or methods according to the present invention, which may be combined with any of the embodiments of the kit of parts or methods preaddressed or still to be addressed unless in contradiction, the hard tissue is a human, animal or artificial hard tissue, optionally a cartilage-covered hard tissue, and optionally wherein the hard tissue is a hard tissue sample outside of the human or animal body, a hard tissue of a dead body or a hard tissue of a human or animal patient or an artificial bone graft material.

Exemplary hard tissues and applications of the kit of parts and methods disclosed herein include:
reinforcement of juvenile bones, in particular correcting deformations by osteotomy or by tethering (e.g. scoliosis of the spine or instable lordosis such as spondylolisthesis, or kyphosis by means of tension belts fastened by suture anchors);
stabilizing of non-unions (non-healing bone fractures), e.g. in juvenile patients by supporting the metaphysis of joints;
stabilizing of bone defects such as osteochondrosis dissecans or perthes; augmentation and stabilization by plate-pin-connections of tumor affected or radiated bone tissue;
ligament or tendon reconstruction in:
   clavicle-shoulder: acromioclavicular reconstruction,
   wrist or knee (medial and lateral tendon reconstruction, augmentation of the patella for the lateral or cranial-caudal translation),
   ankle (lateral or medial ligaments)
open or closed wedge osteotomies to correct deformities and to re-align joints (e.g. knee: tibia plateau, or spine).

In an embodiment of the kit of parts or methods according to the present invention, which may be combined with any of the embodiments of the kit of parts or methods preaddressed or still to be addressed unless in contradiction, the hard tissue has a reduced bone density, optionally is an osteoporotic bone.

In an embodiment of the methods according to the present invention, which may be combined with any of the embodiments of the methods preaddressed or still to be addressed unless in contradiction, the method is for treatment of a shoulder or cruciate ligament injury.

### BRIEF DESCRIPTION OF THE DRAWINGS

Embodiments of the current invention are described in more detail in the following with reference to the figures. These are for illustrative purposes only and are not to be construed as limiting.

FIGS. 1A and 1B show exemplary kits of parts;

FIGS. 2A-2H illustrate recesses in hard tissues and hardening material filled therein and recesses in the hardening material;

FIGS. 3A-3C exemplify how a recess can be created in the hardening material;

FIGS. 4A-4F exemplify how a recess can be created in the hardening material;

FIGS. 5A-5H show examples of implant implantation;

FIGS. 6A-6D show fixation of a tissue with a prosthetic element;

FIGS. 7A-7C show an exemplary device for use in the kit of parts and implantation;

FIG. 8 shows an exemplary device for use in the kit of parts and implantation;

FIG. 9 shows an exemplary device for use in the kit of parts and implantation;

FIGS. 10-10C show exemplary devices for use in the kit of parts and implantation;

FIGS. 11A and 11B show an exemplary device for use in the kit of parts and implantation;

FIGS. 12A and 12B show the use of an implant in a device;

FIGS. 13A and 13B show the use of an implant in a device;

FIGS. 14-14D show an exemplary method using the device;

FIG. 15 a sectional view of liquified parts of an implant (suture anchor) extending from a device into a hardening material in a hard tissue; and

FIGS. 16A-16D a device and sectional views at different magnifications of liquified parts of an implant extending from a device into a hardening material in a hard tissue.

### DETAILED DESCRIPTION

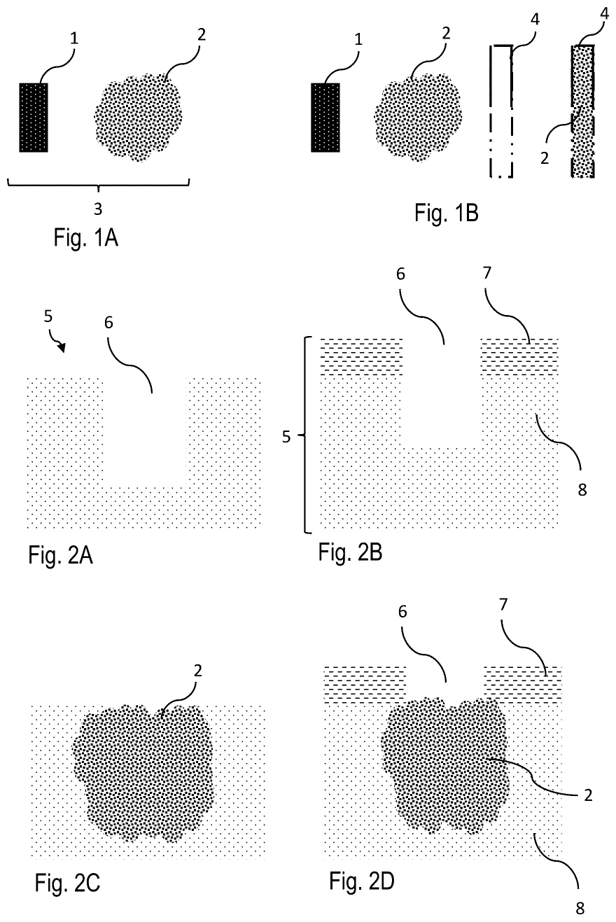

FIG. 1A shows an exemplary kit of parts 3 according to the present invention including an implant 1 having thermoplastic properties and a hardening material (e.g. hydraulic cement) 2, in particular a calcium phosphate-based cement. In all of the following figure descriptions, the term “hardening material” may be used for easier legibility and refers to any hardening materials as disclosed herein, in particular to inorganic materials, biodegradable cements, hydraulic cements, in particular calcium phosphate-based cements. The kit of parts may include the hardening material 2 in powder or neat form, it may be packaged in any suitable way, in particular to extend the shelf life and/or to keep the hardening material (and the implant) sterile. FIG. 1B shows a kit of parts according to the present invention that further includes an instrument 4 configured to administer the hardening material into a recess in a hard tissue (not shown). The instrument 4 can comprise the hardening material 2, and/or it can be a cannula, a punching tool, a drill, a tap or a reamer. The latter four examples can also allow for the instrument 4 to be used to create the recess in the hard tissue, to administer the hardening material 2 and/or to create a recess in the hardening material.

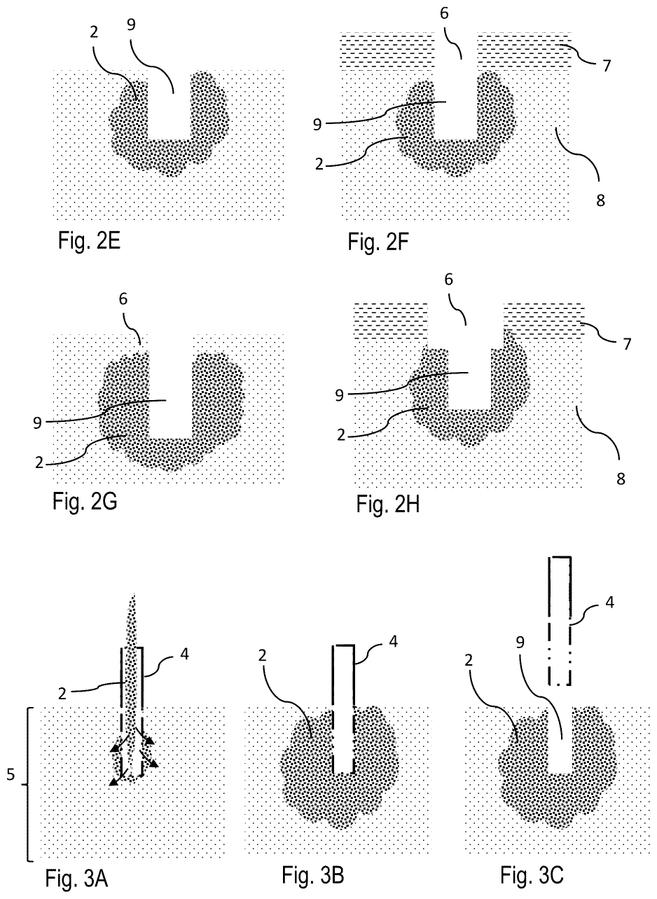

FIG. 2A shows a hard tissue 5 with a recess 6 in the hard tissue. The recess can have any shape and it may also be a passage through the tissue 5 (not shown). The hard tissue may be a spongy bone (trabecular bone). As shown in FIG. 2B, the hard tissue 5 may comprise a cortical bone (part) 7 and a spongy bone (part) 8, wherein the recess 6 in the hard tissue can stretch through the cortical bone 7 into the spongy bone 8. FIG. 2C shows a recess in a hard tissue filled with hardening material 2. The hardening material 2 flows into the porous structure of the bone tissue around the recess though which the hardening material was introduced, where it can harden. FIG. 2D shows an example where the recess 6 reaches through the cortical bone 7 (and before filling with hardening material into the spongy bone 8), while in this example, the hardening material 2 is filled into the recess section in the spongy bone 8 such that it can flow into the spongy bone tissue 8. FIG. 2E illustrates a recess 9 in the hardening material 2 that was filled into the recess in the hard tissue. As shown in FIG. 2F, the recess 9 in the hardening material 2 can, e.g., essentially form a continuous recess with the recess 6 in the hard tissue extending through the cortical bone 7 that was not filled up with hardening material 2. FIG. 2G shows that a part of the recess 6 in the hard tissue may also remain unfilled (e.g. in the spongy bone as shown or in the cortical bone, not shown), and/or that the recess 6 in the hard tissue may be larger in cross-section than the recess 9 in the hardening material 2. Even though not shown, an embodiment, wherein the recess 6 in the hard tissue is smaller in cross section than the recess 9 in the hardening material 2 is also possible. FIG. 2H shows an example, wherein the recess 6 in the cortical bone 7 features a larger cross-section compared to the cross-section 9 in the hardening material 2 that is located in the spongy bone 8.

FIGS. 3A, 3B and 3C show a sequence in which an instrument 4 is used to create a recess in the hard tissue 5 (FIG. 3A). Hardening material 2 can be filled into the recess while the recess is created or after it is created. The instrument is then left in the hardening material 2 (FIG. 3B), for example until the hardening material has hardened sufficiently to become dimensionally stable under standard conditions, as detailed above, and is subsequently pulled out (FIG. 3C) to create the recess 9 in the hardening material. In other words, in this example it is possible to use one instrument 4 to create a recess in the hard tissue, fill the hardening material into the recess and create a recess in the hardening material. Exemplary instruments 4 include a punching tool, a drill, a tap or a reamer that are configured, e.g. by channels, to administer hardening material while creating a recess. In the embodiment shown in the Figures, instrument 4 comprises a multiplicity of openings allowing for dispensing the hardening material. However, also instruments comprising only one or two of such openings, or slots or other geometrical forms can be used.

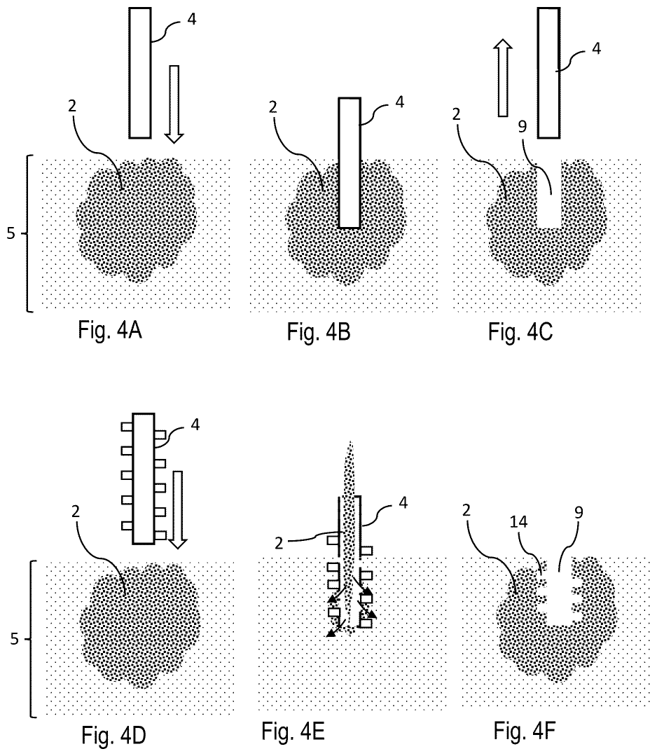

FIGS. 4A, 4B and 4C show an alternative sequence for creating a recess 9 in the hardening material 2. In this example, the hardening material is (already) in the hard tissue (e.g. filled into recess therein, FIG. 4A) and an instrument 4 is used to create a recess 9 in the hardening material 2, e.g. either by pushing the instrument into the hardening material 2 while it is still pasty or liquid, or by screwing, drilling or punching into the hardening material 2 (FIG. 4B). In the former case, the instrument 4 can be any suitably shaped object, including a needle, a screw or a cannula, and in the latter case a suitable tool can be a punching tool, a screw, a drill, a tap or a reamer. Upon removal of the instrument 4, the recess 9 in the hardening material 2 is freed and can be accessed, e.g. by the implant of the present invention (FIG. 4C).

FIGS. 4D, 4E and 4F show how a recess 9 in the hardening material 2 can be created by the instrument 4 either when the hardening material is already in the hard tissue 5 (FIG. 4D) or while filling the hardening material 2 into the hard tissue with the instrument 4 (FIG. 4E) while a pattern (e.g. cut-outs or a thread) 14 is created in the recess 9 (FIG. 4F) by suitable means on the instrument 4, e.g. by the instrument being a tap or reamer.

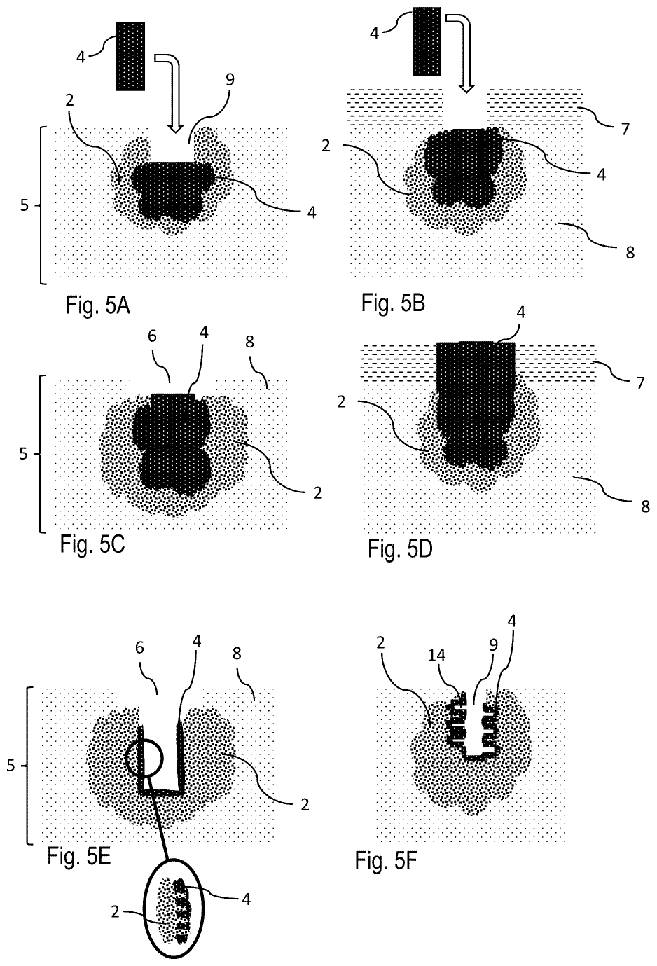

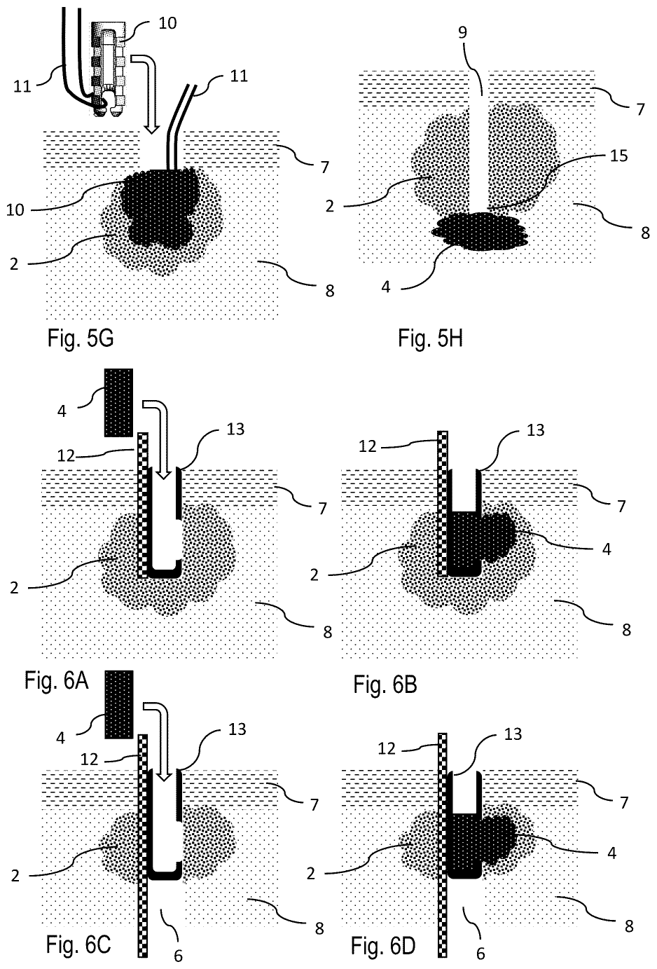

FIGS. 5A to 5H show examples of how an implant 4 having thermoplastic properties as described herein can be inserted and implanted into a recess 9 in the hardening material 2. As shown in FIG. 5A, the implant 4 can be inserted into a hard tissue with a recess that is at least partially filled with a (optionally hardened) hardening material, wherein the recess in the hard tissue and the recess 9 in the hardening material may align to essentially form one recess. During or after implantation, vibrational energy is transferred to the implant 4 which causes the implant to at least partially liquify. The liquified part(s) of the implant 4 flow into the pores or cracks of the hardening material 2 (e.g. not the entire recess 9 may be filled) which leads to a reinforcement of the hardening material's structure, a strong retention of the implant due to the large surface area into which the implant 4 can flow into and/or a reduction of the brittleness of the hardening material (the implant resolidifies upon cooling, which is clear to the skilled person and therefore not continuously mentioned herein). FIGS. 5B and 5C show examples in which the implant 4, after liquification and resolidification, fills the entire recess in the hardening material 2. Alternatively, and as shown in FIG. 5D, the liquified and resolidified implant 4 may also fill the recess in the hard tissue, e.g. up to the entry point of the recess in the hard tissue, e.g. the cortical bone 7. FIG. 5E an exemplary embodiment where the resolidified implant 4 is located on parts of the recess walls where the implant 4 makes a form fit with the surface of the recess 9 in the hardening material 2. The walls of recess 9, under close-up inspection (circle), may not be entirely smooth but comprise pores, indentations or the like with which the implant 4 forms a form fit. Of course, this close-up also applies to hardening material-implant interactions in examples in which the implant fills the volume of the recess (i.e. in FIGS. 5A-5D). FIG. 5F exemplifies how a pattern 14 is formed in the recess 9 in the hardening material 2, wherein the liquified and resolidified implant 4 form fits with the pattern. The implant could also fill a part or the entire volume of the recess 9 (not shown). FIG. 5G shows an example in which a suture anchor 10 is used as an implant which comprises a suture 11 threaded through a suitable opening in the suture anchor. The liquified part(s) of the implant 4 flow into the pores or cracks of the hardening material 2 and stably encompass and fixate the suture 11 in the recess in the hard tissue. FIG. 5H shows an example in which the implant 4 was guided through the recess 9 in the hardening material 2 resulting in fixation of the implant 4 in an area of the hard tissue adjacent to the distal end 15 of the recess 9 and optionally at least partly in the recess 9 in the hardening material 2. This example can be used, e.g. with a suture anchor as an implant, in which case, for example, a suture (not shown) can be fixated with the implant and guided through the recess 9 out of the hard tissue.

FIGS. 6A to 6D show examples of the use of the implant 4 and hardening material 2 for the fixation of a tissue 12 (or any other object) with a corresponding prosthetic element 13. A prosthetic element 13 (e.g. as described in WO 2011/091545) can be used to create a press-fit in a recess in hardening material 2 (and optionally in a recess in a hard tissue) with an object, e.g. tissue 12 (FIG. 6A). The element 13 can comprise a (inner) void and a passage from the void to the outside of the element such that when the implant is inserted into the element 13 and is at least partially liquified, the liquified part(s) of the implant flow out from the element through the passage and into the hardening material 2 (FIG. 6B). This leads to a fixation of the element 13, a structural strengthening of the hardening material, and thereby a stable fixation of the object, e.g. tissue 12. As shown in FIGS. 6C and 6D, the recess 6 in the hard tissue may also extend through the hard tissue and the fixation is achieved analogously to the example described in FIGS. 6A and 6B.

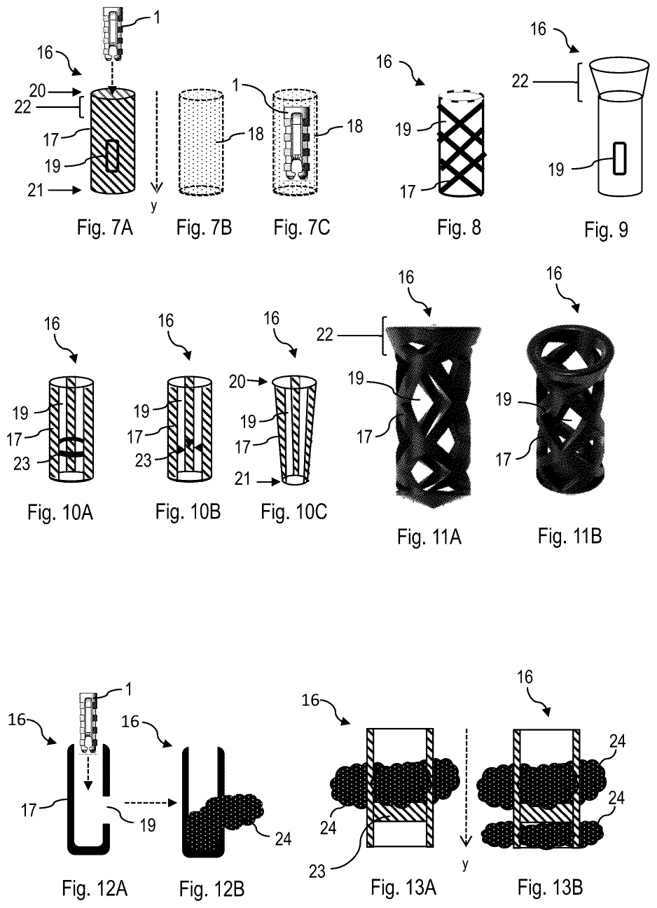

FIGS. 7A to 7C exemplify a device 16 for use in the kit of parts or methods described herein, which device can be an element as described for FIG. 6, but typically differs from known elements such as prosthetic elements in that it is not necessarily dimensionally stable and/or is suitable for use in the absence of press-or positive-fit connections because it can be used in the non-hardened or partially hardened hardening material, where it serves as reinforcement means and/or as a tool to liquify and direct liquified parts of the implant described herein into the non-hardened or partially hardened hardening material, which itself may not provide sufficient resistance for liquifying the implant directly. As shown in a side view in FIG. 7A, the device 16 has a body 17 and at least one aperture 19 in the body, in particular between a proximal end 20 and a distal end 21 of the body, e.g. in the sides of the body. The device can have any geometrical shape including a cylindrical, cube, cuboid, cone, pyramid or prism shape, wherein the prism can be polygonal, and it extends from a proximal end 20 to a distal end 21 along a y-axis. At the distal end 20 or in a proximal section 22, the body has an opening for inserting the implant 1 into the device, e.g. while attached to a sonotrode, specifically into an inner volume 18 of the body shown in FIGS. 7B and 7C as an imaginary structure. When applying ultrasound to the implant 1, during or after inserting the implant into the device 16, the implant at least partially liquified and the liquified parts of the implant can exit the inner volume 18 of the device 16 via the at least one aperture 19. The site at which the liquified parts of the implant exit the device can be adjusted by positioning the aperture 19, and of course more than one aperture can be installed in the device. The device 16 and body 17 can be made of titanium, steel, zinc, magnesium, alloys thereof, calcium phosphate ceramics, in particular hydroxyapatite, high-performance plastics, in particular polyether ether ketone (PEEK), polyetherketoneketone (PEKK), polyaryletherketone (PAEK), polysulfones (PSU) or polyphenylene sulfides (PPS).

FIG. 8 shows a side view of an exemplary device 16 having a net-shaped body 17, wherein the voids in the net form the apertures 19. Generally, the body (in any shape or form) of the device can be compressible, in particular regarding its cross-section. For example, a net-shape can be suitable for conveying compressible properties. Alternatively, or additionally, the body may have one or more suitable slots extending between the proximal and distal ends of the body which allow for compression, and/or be formed of a compressible material.

As shown in FIG. 9, the proximal section 22 can be formed as or comprise a collar, in particular a collar tapered with its widest cross-section at the proximal end. The collar can be suitable, e.g., to rest against a surface of a recess in a hard tissue, e.g. to provide a good seating of the device and/or to protect a suture extending out from the inner volume of the device against the surrounding tissue, or to protect the tissue against the suture, e.g. from irritation or “cutting in”. The collar may also have a thread on its outer circumference, or other means suitable for fixating the collar to surrounding tissue.

When applying ultrasound to the implant 1, during or after inserting the implant into the device 16, the implant at least partially liquifies and the liquified parts of the implant can exit the inner volume 18 of the device 16 via the at least one aperture 19. To assist and/or direct the location of the liquification relative to the device, the device 16 may be configured to cause a resistance against the implant being inserted into the device, and/or comprise means configured to cause a resistance as shown in FIGS. 10A, 10B and 10B. FIG. 10A shows an example in which the means 23 have a ring-like structure running around the inner side of the body 17, i.e. inside the inner volume and connecting elements forming the body 17. The means 23 can be positioned at the same position along the y-axis or at different positions. In this example, the apertures 19 are formed between elements forming the body 17, however, the body may also be an essentially closed structure with one ore more aperture(s) as shown in FIG. 7A. FIG. 10B shows an example for the means 23 in which these are configured as protrusions extending from the body 17 into the inner volume (three are shown, but any number is possible depending on the specific application). The means 23 can be positioned at the same position along the y-axis or at different positions. FIG. 10C shows an example of how the device 16, in particular the body 17, can be configured to cause a resistance against the implant being inserted into the inner volume along the y-axis. The body 17 (or the entire device) can have a tapered or cone shape, wherein the diameter of the inner volume is reduced in direction of insertion of the implant along the y-axis towards the distal end 21 of the body 17. At some point along the y-axis, the diameter of the implant and the diameter of the body will be of the same size causing a resistance against the implant being inserted into the inner volume and liquification of the implant starting at the site of resistance (where the implant contacts the body member). Referring back to FIG. 8, the means configured to cause a resistance can be formed by the net shaped body, in particular by body parts or sections that have a horizontal component (the y-axis being considered the vertical axis).

FIGS. 11A and 11B respectively show a side and a side/top view of an exemplary device 16 disclosed herein. The body 17 can be net-shaped and include the apertures 19. Due to the shape, the body 17, in particular parts or sections thereof, can also act as means for creating a resistance. For example, the device can be made from titanium.

FIGS. 12A and 12B exemplify, in a side view, how the implant 1 can be inserted into the device 16 having an aperture 19 in the body 17. The implant is inserted, e.g., via an opening in the proximal section or at the proximal end into the device (FIG. 12A). Upon application of ultrasound to the implant 1, at least a part thereof liquifies and the liquified parts 24 can exit the device through the aperture (FIG. 12B).

FIGS. 13A and 13B essentially show what was described for FIGS. 12A and 12B, with the addition of means for causing a resistance 23, which initiate liquification an inserted implant by causing a resistance against the implant, e.g. and edge causing resistance against the implant (concentration of energy). The liquified part(s) of the implant 24 then extend out of the inner volume of the body of the device 16 to an outside of the device through the apertures. Depending on, e.g., the volume of the implant, the size of the device 16, the amount of energy transferred to the implant and/or the speed of insertion of the implant, the liquified implant 24 may also flow further in direction of the distal end of the body where it can also extend out from the device through (optional) further apertures, as shown in FIG. 13B.

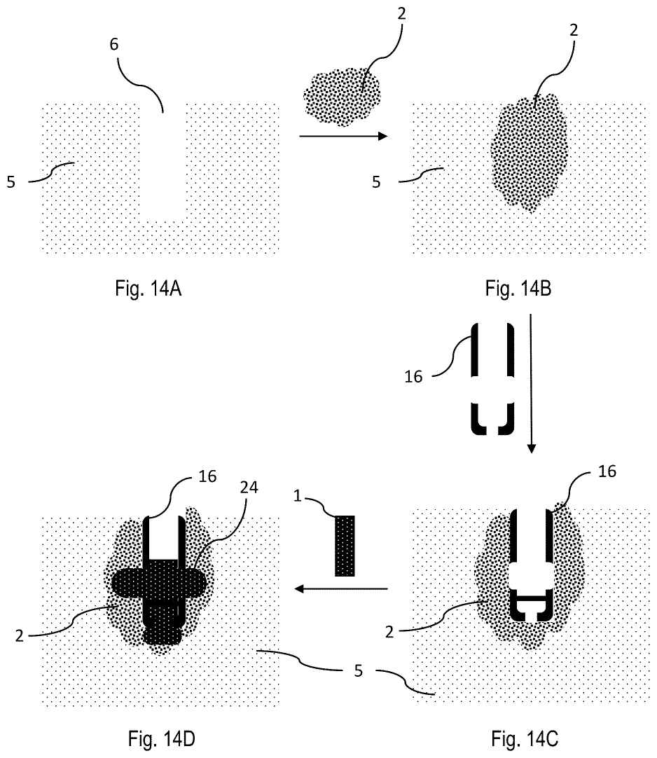

FIGS. 14A to 14D show an example of the methods described herein for implanting an implant into a hard tissue with the device 16, in particular when using a kit of parts as described herein or when using the components of the kit of parts individually. As shown in FIG. 14A, a hard tissue 5 with a recess 6 is provided, or a hard tissue is provided and a recess is created, optionally by drilling (not shown). The recess 6 is subsequently at least partially filled with the hardening material 2 for orthopedic use (FIG. 14B). A device 16 as described herein is provided and inserted into the hardening material 2. This step is preferably performed while the hardening material is still non-hardened or partially hardened, i.e. has a viscosity which allows for inserting the device into the hardening material without drilling, e.g. just by pushing it in. Alternatively, the hardening material can be hardened and a recess in the hardening material can be formed during filling (e.g. by means of the instrument used for filling) or after filling (e.g. by drilling) the material into the recess in the hard tissue, wherein the recess in the hardening material is suitable for inserting the device (not shown). As shown in FIG. 14C, the implant having thermoplastic properties 1 is inserted into the device 16 while or followed by transmitting ultrasound to the implant (not shown, e.g. with a sonotrode attached to the implant) to liquify the implant to the extent that a liquified at least partial volume 24 of the implant extends out of the inner volume of the device through the at least one aperture. This step is preferably performed while the hardening material is still non-hardened or partially hardened, i.e. has a viscosity which allows liquified parts 24 of the implant to displace the hardening material while flowing out of the device, e.g. while forming, e.g., roundish and/or soft-edged shapes of liquified and subsequently re-solidified implant material 24 which aids in retaining the implant and reduce stress due to the roundish shape. Alternatively, the hardening material can be hardened when the implant 1 is inserted into the device 16 and at least partially liquified so that the implant can flow into pores or cracks of the hardening material, wherein pores or cracks can refer to a “rough” surface of the hardening material where the implant material creates a form fit.

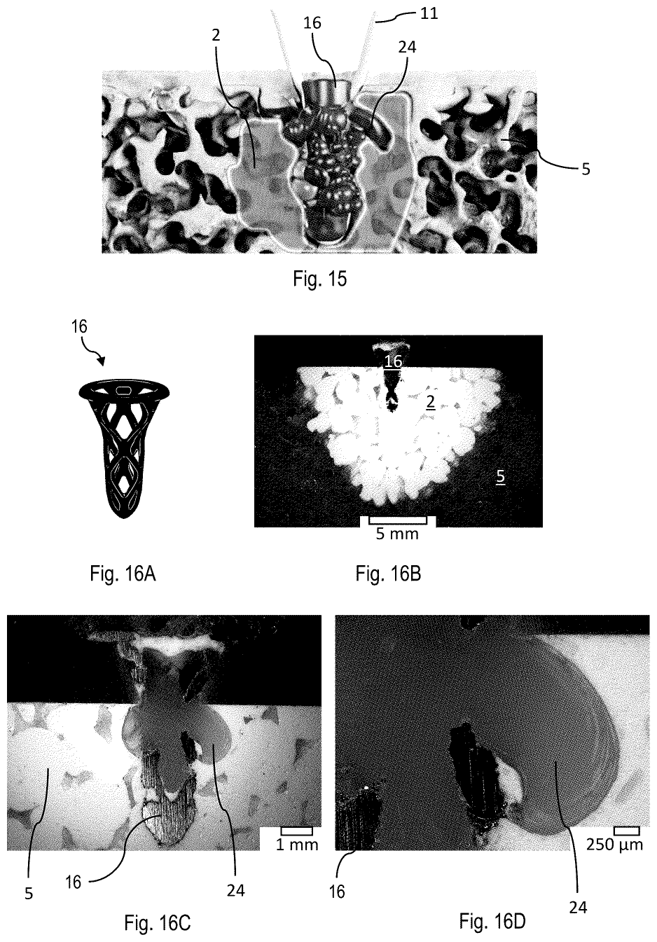

FIG. 15 shows a sectional view of an implant (here, for example, a suture anchor with suture 11) and a device 16 inserted into a hardening material 2 which was filled into a recess in a hard tissue 5. The implant was inserted into the device 16 through an opening of the device 16 into the inner volume of the device while or followed by transmitting ultrasound (e.g. with a sonotrode) to the implant to liquify the implant to the extent that a liquified at least partial volume 24 of the implant extends out of the inner volume of the device through the at least one aperture of the device 16 into the hardening material 2. For example, the insertion of the device 16 into the hardening material 2 and the insertion of the implant into the device 16 may have been carried out while the hardening material 2 was non-hardened or partially hardened. Alternatively, the device 16 may have been inserted into a recess in the hardened (or partially hardened) hardening material 2, and the insertion of the implant into the device 16 may have been carried out when the hardening material 2 was hardened.

FIGS. 16A to 16D show a device and an implant inserted into a hardening material 2 which was filled into a recess in a hard tissue 5. FIG. 16A shows an exemplary device 16 having a net-shaped body, wherein the voids in the net form the apertures. This device 16 is the device depicted in the sectional views of FIGS. 16B to 16D, however, any other device as described herein can be used. FIG. 16B shows a sectional view at a first magnification of an implant and a device 16 inserted into a hardening material 2 which was filled into a recess in a hard tissue 5. The implant was inserted into the device 16 through an opening of the device 16 into the inner volume of the device while or followed by transmitting ultrasound (e.g. with a sonotrode) to the implant to liquify the implant to the extent that a liquified at least partial volume (not visible in this magnification) of the implant extends out of the inner volume of the device through the at least one aperture of the device 16 into the hardening material 2. FIG. 16C shows an excerpt of FIG. 16B at a second, greater magnification. It can be seen that a liquified at least partial volume 24 of the implant extends out of the inner volume of the device through the at least one aperture of the device 16 into the hardening material 2. In this example, the insertion of the implant into the device 16 while or followed by transmitting ultrasound (e.g. with a sonotrode) to the implant was carried out while the hardening material 2 was non-hardened or partially hardened. The liquified parts 24 of the implant displaced the non-hardened or partially hardened hardening material 2 while forming roundish and soft-edged shapes of liquified and subsequently re-solidified implant material 24 which aids in retaining the implant and reduce stress due to the roundish shape. FIG. 16D shows an excerpt of FIG. 16C at a third, greater magnification, further detailing how the liquified parts 24 of the implant displaced the non-hardened or partially hardened hardening material 2 while forming roundish and soft-edged shapes of liquified and subsequently re-solidified implant material 24

### REFERENCE SIGNS LIST

1 Implant
2 Hardening material (e.g. hydraulic cement)
3 Kit of parts
4 Instrument
5 Hard tissue
6 Recess in hard tissue
7 Spongy bone
8 Cortical bone
9 Recess in hardening material
10 Suture Anchor
11 Suture
12 Tissue or object
13 Prosthetic element
14 Pattern (e.g. cut-outs or thread)
15 Distal end of recess
16 Device
17 Body
18 Inner volume
19 Aperture
20 Proximal end
21 Distal end
22 Proximal section
23 Means for causing a resistance
24 Liquified/Resolidified implant

1. A kit of parts for implanting an implant into a hard tissue, the kit of parts comprising:
a hardening material for orthopedic use, and
an implant having thermoplastic properties.

2. The kit of parts according to claim 1, wherein the kit of parts further comprises an instrument configured to administer the hardening material into a recess in a hard tissue.

3. The kit of parts according to claim 2, wherein at least one of: the instrument is further configured to create a recess in a structure formed by the hardening material into which the implant is configured to be implanted, the instrument is further configured to create a recess in a hard tissue, or a combination thereof.

4. The kit of parts according to claim 1, wherein the kit of parts further comprises a tool for creating a recess in a hard tissue and/or for creating a recess in a structure formed with the hardening material, into which structure the implant can be implanted.

5. The kit of parts according to claim 2, wherein the instrument is a cannula, a punching tool, a drill, a tap, a screw or a reamer.

6. The kit of parts according to claim 2, wherein the instrument is a screw, a tap or a reamer configured to administer the hardening material into a recess in a hard tissue, and to create a recess in a structure formed by the hardening material into which the implant can be implanted.

7. The kit of parts according to claim 1, wherein the kit further comprises reinforcement means for the hardening material.

8. The kit of parts according to claim 1, wherein the kit further comprises a device having a body defining an inner volume, wherein
the device is shaped and dimensioned to receive the implant in the inner volume,
the body of the device comprises at least one aperture,
the device is configured such that upon transmission of ultrasound energy to the implant during or after insertion of the implant into the inner volume, a liquified at least partial volume of the implant extends out of the inner volume through the at least one aperture.

9. The kit of parts according to claim 8, wherein
the body extends from a proximal end to a distal end along a y-axis,
a proximal section of the body forms an opening for inserting the implant into the inner volume, and
the at least one aperture is positioned between the opening and the distal end of the body.

10. The kit of parts according to claim 9, wherein at least one of:
the proximal section of the body is or comprises a collar,
the body is a net-shaped body,
the body is compressible,
or a combination thereof.

11. The kit of parts according to claim 1, wherein at least one of:
the implant is configured to be at least partially liquified by ultrasound,
the kit of parts further comprises a sonotrode,
or a combination thereof.

12. The kit of parts according to claim 1, wherein at least one of:
the implant is a pin, an implant for tenodesis or a suture anchor,
the kit further comprises a suture for use with the suture anchor,
or a combination thereof.

13. A method for implanting an implant into a hard tissue, the method comprising:
(a1) providing a hard tissue with a recess that is at least partially filled with a hardened hardening material for orthopedic use, wherein the hardened hardening material comprises a recess, and inserting an implant having thermoplastic properties into the recess in the hardened hardening material while or followed by transmitting ultrasound to the implant for a time sufficient to liquify the implant to an extent that liquification results in fixation of the implant in the recess in the hardening material
or
(a2) providing a hard tissue with a recess that is at least partially filled with a non-hardened, partially hardened or hardened hardening material for orthopedic use, followed by creating a recess into the hardened hardening material and inserting an implant having thermoplastic properties into the recess in the hardened hardening material while or followed by transmitting ultrasound to the implant for a time sufficient to liquify the implant to the extent that liquification results in fixation of the implant in the recess in the hardening material, or followed by inserting an implant having thermoplastic properties into the non-hardened or partially hardened hardening material while or followed by transmitting ultrasound to the implant for a time sufficient to liquify the implant to the extent that liquification results in fixation of the implant in the hardening material and hardening the hardening material,
or
(a3) providing a hard tissue with a recess that is at least partially filled with a non-hardened or partially hardened hardening material for orthopedic use, followed by providing a device having a body defining an inner volume and comprising at least one aperture, inserting the device into the hardening material, and inserting an implant having thermoplastic properties through an opening of the device into the inner volume of the device while or followed by transmitting ultrasound to the implant to liquify the implant to the extent that a liquified at least partial volume of the implant extends out of the inner volume of the device through the at least one aperture, wherein the insertion of the device into the hardening material and the insertion of the implant into the device is carried out while the hardening material is non-hardened or partially hardened.

14. A method for implanting an implant into a hard tissue, the method comprising:
(a) providing a kit of parts according to claim 1,
(b1) providing a hard tissue with a recess, or
(b2) providing a hard tissue and creating a recess into the hard tissue,
(c) filling the recess at least partially with a hardening material for orthopedic use,
(d1) at least partially hardening the hardening material and creating a recess into the hardened or partially hardened hardening material, followed by inserting the implant into the recess in the hardening material while or followed by transmitting ultrasound to the implant for a time sufficient to liquify the implant to an extent that liquification results in fixation of the implant in the recess in the hardening material, or
(d2) inserting the implant into non-hardened or partially hardened hardening material while or followed by transmitting ultrasound to the implant for a time sufficient to liquify the implant to the extent that liquification results in fixation of the implant in the hardening material and hardening the hardening material, or
(d3) providing a device having a body defining an inner volume and comprising at least one aperture, inserting the device into the hardening material, and inserting the implant through an opening of the device into the inner volume of the device while or followed by transmitting ultrasound to the implant to liquify the implant to the extent that a liquified at least partial volume of the implant extends out of the inner volume of the device through the at least one aperture,
wherein the insertion of the device into the hardening material and the insertion of the implant into the device is carried out while the hardening material is non-hardened or partially hardened.

15. A method for implanting an implant into a hard tissue, wherein the method comprises:
(a) providing the kit of parts,
(b1) providing a hard tissue with a recess, or
(b2) providing a hard tissue and creating a recess into the hard tissue,
(c) filling the recess at least partially with the hardening material and creating a recess into the hardening material before or after hardening of the hardening material, wherein the recess in the hardening material has a proximal side facing outside of the hard tissue and a distal side facing interior of the hard tissue, and
guiding the implant through the recess in the hardening material while or followed by transmitting ultrasound to the implant for a time sufficient to liquify the implant to the extent that liquification results in fixation of the implant in an area of the hard tissue adjacent to a distal end of the recess in the hardening material.

16. The method according to claim 13, wherein:
the recess in the hardening material is created by inserting an instrument into the non-hardened or partially hardened hardening material in the recess of the hard tissue, or
the recess in the hardening material is created by inserting an instrument into the non-hardened or partially hardened hardening material in the recess of the hard tissue, wherein the instrument is further used for at least partially filling the recess in the hard tissue with the hardening material.

17. The method according to claim 14, wherein at least one of: the creating of the recess in the hard tissue, the filling of the recess with the hardening material, the creating of the recess in the hardening material, or a combination thereof is performed with a same instrument.

18. The method according to claim 13, wherein at least one of the implant, the recess in the hardening material, or both is/are configured such that at least one of: a liquified part of the implant makes a form fit with at least a part of the hardening material, the liquified part of the implant fills pores in the recess, the liquified part of the implant fills an entire recess, or a combination thereof, when inserting the implant into the recess in the hardening material while or followed by transmitting ultrasound to the implant for a time sufficient to liquify the implant to the extent that liquification results in fixation of the implant in the recess in the hardening material.

19. The method according to claim 13, wherein the implant is an implant for tenodesis or a suture anchor that comprises a suture threaded through a suture conduit and optionally wherein the method further comprises threading the suture through the suture conduit of the suture anchor.

20. The method according to claim 13, wherein the implant is a suture anchor that comprises a suture threaded through a suture conduit and optionally wherein the method further comprises threading the suture through the suture conduit of the suture anchor and the method further comprises attaching a tissue, optionally a ligament, with the suture of the suture anchor to the hard tissue, before or after insertion of the implant into the recess in the hardening material.

21. The method according to claim 14, wherein instrument for at least partially filling the recess in the hard tissue with the hardening material and/or creating the recess in the hardening material comprises an object to be implanted, wherein during the filling and/or creating of the recess, the object is implanted and the instrument is subsequently removed.

22. The method according to claim 14, wherein the method further comprises before the filling the recess in the hard tissue with the hardening material, inserting reinforcement means for the hardening material, in particular tubular reinforcement means, in particular a porous or woven tube, followed by filling the recess comprising the reinforcement means with the hardening material.

23. The method according to claim 13, wherein the method further comprises at least one of:
inserting a tissue, optionally a ligament, into the recess in the hardening material before or during insertion of the implant into the recess in the hardening material;
inserting a further prosthetic element into the recess in the hardening material before or during insertion of the implant into the recess in the hardening material, optionally a prosthetic element that has a press-fit in the recess; or
a combination thereof.

24. The method according to claim 13, wherein at least one of: the hard tissue is a human, animal or artificial hard tissue, the hard tissue has a reduced bone density, or a combination thereof.

25. The method according to claim 13, wherein the method is for treatment of a shoulder or cruciate ligament injury.

26. The method according to claim 14, wherein at least one of:
a proximal section of the body of the device is or comprises a collar,
the body of the device is a net-shaped body,
the body of the device is compressible,
or a combination thereof.
